(19) United States
(12) Patent Application Publication
(10) Pub. No.: US 2024/0423605 A1
(43) Pub. Date: Dec. 26, 2024

(54) ANCHORING DEVICE AND ANCHORING APPARATUS

(71) Applicant: MICRO-TECH (NANJING) CO., LTD., Nanjing (CN)

(72) Inventors: Kenneth BINMOELLER, Rancho Santa Fe, CA (US); Yimin Sun, Nanjing (CN); Jianyu Wei, Nanjing (CN); Qi Lin, Nanjing (CN); Jiefeng Xi, Nanjing (CN); Zhenghua Shen, Nanjing (CN); Changqing Li, Nanjing (CN); Derong Leng, Nanjing (CN)

(21) Appl. No.: 18/824,584

(22) Filed: Sep. 4, 2024

Related U.S. Application Data
(63) Continuation-in-part of application No. PCT/CN2023/082641, filed on Mar. 21, 2023.
(60) Provisional application No. 63/321,820, filed on Mar. 21, 2022.

(51) Int. Cl. A61B 17/04; A61B 17/00

(52) U.S. Cl. CPC A61B 17/0401; A61B 2017/00818; A61B 2017/0409

(57) ABSTRACT

Disclosed are an anchoring device and an anchoring apparatus. The anchoring device includes a coupling member and two anchoring members. The two anchoring members are arranged on the coupling member at intervals in a first direction. A dimension of the anchoring member in a second direction is a first dimension, the second direction is perpendicular to the first direction. The anchoring member is configured to switch between a first configuration and a second configuration. When the anchoring member is in the first configuration, a dimension of the anchoring member in a direction perpendicular to the first direction is a second dimension. When the anchoring member is in the second configuration, a dimension of the anchoring member in the second direction is a third dimension, and the third dimension is greater than the second dimension and the first dimension.

### CROSS-REFERENCE TO RELATED APPLICATIONS

This application is a continuation-in-part of International Application No. PCT/CN2023/082641, filed on Mar. 21, 2023, which claims priority of a provisional application No. 63/321,820, filed with United States Patent and Trademark Office on Mar. 21, 2022, titled “SYSTEMS AND DEVICES FOR GASTROINTESTINAL PROCEDURES”. Both of the aforementioned applications are hereby incorporated by reference in their entireties.

### TECHNICAL FIELD

The present application relates to the technical field of medical apparatus, and in particular, to an anchoring device and an anchoring apparatus.

### BACKGROUND

In general, the abdominal cavity is separated from the thoracic (e.g., chest) cavity by a diaphragm. FIG. 1 is a schematic diaphragm of the intersection of an abdominal cavity and a thoracic cavity. The diaphragm 101 separates the abdominal cavity (positioned below the diaphragm 101) from the thoracic cavity (positioned above the diaphragm 101). The diaphragm 101 is a membranous muscle that aids in respiration and causes the thoracic cavity to enlarge during inhalation and contract during exhalation. An esophagus 102 extends from the thoracic cavity, passes through an esophageal hiatus in the diaphragm, and couples with a gastric cavity 103. A transition area 104 connecting the stomach and the esophagus is called the cardiac orifice. Under normal circumstances, the cardiac orifice will be narrowed under an influence of the esophageal sphincter, close a passage between the gastric cavity and the esophagus, and form an anti-reflux barrier to prevent reflux of stomach contents. The diaphragm 101 has crura of diaphragm at the esophageal hiatus, which is divided into right crus of diaphragm 106 and left crus of diaphragm 107, in which the left limb of right crus of diaphragm 106 wraps outside a junction of the esophagus 102 and the gastric cavity 103 to fix positions of the gastric cavity and the esophagus, so that a certain angle is formed between gastric fundus and esophageal cardia, which is clinically called His angle. In a human body, the esophageal sphincter, His angle and the crura of diaphragm together form a set of anti-reflux mechanism to prevent stomach contents from flowing back into the esophagus.

When the crus of diaphragm are loose, it cannot fix the positions of the esophagus and the gastric cavity. Under an effect of intra-abdominal pressure, the gastric cavity 103 will slide into the thoracic cavity through the esophageal hiatus in the diaphragm to form a hiatus hernia. At this time, the right crus of diaphragm can no longer wrap around the cardiac orifice well, His angle will disappear, the anti-reflux mechanism will become disabled, and the stomach contents are easy to form reflux, causing GERD (Gastro-esophageal Reflux Disease).

### SUMMARY

The present application discloses an anchoring device. The anchoring device can be configured for a gastrointestinal procedure. For example, it is configured to secure the gastric wall, diaphragmatic muscle, and esophageal wall to restore the anti-reflux function at the junction of esophagus and stomach.

In some aspects, the present application provides an anchoring device, including a coupling member and two anchoring members. The two anchoring members are arranged on the coupling member at intervals along a first direction. A dimension of the coupling member in a second direction is a first dimension, and the second direction is perpendicular to the first direction. The anchoring member is configured to switch between a first configuration and a second configuration. When the anchoring member is in the first configuration, a dimension of the anchoring member in a direction perpendicular to the first direction is a second dimension. When the anchoring member is in the second configuration, a dimension of the anchoring member in the second direction is a third dimension, the third dimension is larger than the second dimension and the first dimension.

In the anchoring device provided above, the coupling member penetrates through a target tissue, the coupling member may abut at two opposite sides of the target tissue by two anchoring members respectively to secure the target tissue, thereby achieving a purpose of correcting morphology of the target tissue.

In some embodiments, one application scenario of the anchoring device provided by the present application is for a gastrointestinal procedure. Exemplarily, among the two anchoring members of the anchoring device, one is a gastric anchoring member and the other is an esophageal anchoring member. Where, the gastric anchoring member is configured to extend through the gastric wall in a first gastric configuration and abuts the gastric wall in a second gastric configuration; the esophageal anchoring member is configured to extend through a delivery catheter in a first esophageal configuration and abuts the esophageal wall in a second esophageal configuration. The coupling member is positioned between the gastric anchoring member and the esophageal anchoring member and coupled with the gastric anchoring member at a first end and coupled with the esophageal anchoring member at a second end. Where, the first gastric configuration is a gastric configuration that is not restricted by the anchoring device. The second gastric configuration is a gastric configuration that is restricted by the anchoring device. The first esophageal configuration is an esophageal configuration that is not restricted by the anchoring device. The second esophageal configuration is an esophageal configuration that is restricted by the anchoring device.

In some embodiments, a length of the coupling member is adjustable to allow the anchoring device to adapt to a difference in tissue thickness between different target tissue structures. In other embodiments, the length of the coupling member is fixed. The length of the coupling member is set according to a distance between the esophageal wall and the gastric wall to allow the gastric anchoring member to abut the gastric wall and the esophageal anchoring member to abut the esophageal wall.

In some embodiments, the gastric anchoring member is configured to extend through the gastric wall in the first gastric configuration and abut the gastric wall in the second gastric configuration; the esophageal anchoring member is configured to extend through the delivery catheter in the first esophageal configuration and abut the esophageal wall in the second esophageal configuration; and the coupling member is positioned between the gastric anchoring member and the esophageal anchoring member and coupled with the gastric anchoring member at the first end, and coupled with the esophageal anchoring member at the second end. Where, prior to abutting the gastric wall, the first gastric configuration and the coupling member have a first gastric angular relationship; and after abutting the gastric wall, the second gastric configuration and the coupling member have a second gastric angular relationship.

In some embodiments, prior to abutting the esophageal wall, the first esophageal configuration and the coupling member have a first esophageal angular relationship; and after abutting the gastric wall, the second esophageal configuration and the coupling member have a second esophageal angular relationship.

In some aspects, the present application provides an anchoring apparatus. The anchoring apparatus includes a delivery device and the anchoring device disclosed above. The delivery device includes a delivery catheter, the anchoring device is movably arranged in the delivery catheter, and the anchoring device is configured to pass through from one end of the delivery catheter. In a case that the anchoring device is positioned within the delivery catheter, the anchoring member is in a first configuration; in a case that the anchoring device is positioned outside the delivery catheter, the anchoring member is in a second configuration.

In the anchoring apparatus provided above, an end of the delivery device that is configured to penetrate through a target tissue is a distal end, and an end far away from the target tissue is a proximal end. First, the anchoring device is movably arranged within the delivery catheter to cause the anchoring device to be close to the distal end of the delivery catheter. For two anchoring members of the anchoring device, an anchoring member close to the proximal end of the delivery catheter is a proximal-end anchoring member, and an anchoring member far away from the proximal end of the delivery catheter is a distal-end anchoring member. Further, the delivery catheter penetrates through a medial wall of an inner cavity of a first target tissue to an inner cavity of a second target tissue, so that the distal-end anchoring member can be delivered to the inner cavity of the second target tissue through the delivery catheter. Then, the distal-end anchoring member is first released, so that the distal-end anchoring member is able to be switched from the first configuration to the second configuration, and then the distal-end anchoring member may abut the medial wall of the inner cavity of the second target tissue. Further, the delivery device is moved, and the proximal-end anchoring member is released when the proximal-end anchoring member moves into the inner cavity of the first target tissue, so that the proximal-end anchoring member may be switched from the first configuration to the second configuration, and then the proximal-end anchoring member may abut the medial wall of the inner cavity of the first target tissue.

The configuration of the present application, its other inventive purposes and beneficial effects will be more obvious and understandable through the description of the preferred embodiments in combination with the drawings.

### BRIEF DESCRIPTION OF DRAWINGS

FIG. 1 is a schematic diagram of an intersection of an abdominal cavity and a thoracic cavity.

FIG. 2 is a schematic diagram of a delivery catheter penetrating a target tissue in some embodiments.

FIG. 3 is a schematic diagram of a distal-end anchoring member of an anchoring device in a second configuration in some embodiments.

FIG. 4 is a schematic diagram of a proximal-end anchoring member of an anchoring device in a second configuration in some embodiments.

FIG. 5 is a first schematic diagram of an anchoring device and a target tissue that are fixed together in some embodiments.

FIG. 6 is a second schematic diagram of an anchoring device and a target tissue that are fixed together in some embodiments.

FIG. 7 is a first schematic diagram of an anchoring device in some embodiments.

FIG. 8 is a second schematic diagram of an anchoring device in some embodiments.

FIG. 9 is a third schematic diagram of an anchoring device in some embodiments.

FIG. 10 is an enlarged schematic diagram at A in FIG. 9.

FIG. 11 is a fourth schematic diagram of an anchoring device in some embodiments.

FIG. 12 is a schematic diagram of an anchoring device coupling with a piercing head in some embodiments.

FIG. 13 is a fifth schematic diagram of an anchoring device in some embodiments.

FIG. 14 is a sixth schematic diagram of an anchoring device in some embodiments.

FIG. 15 is an assembly diagram of a delivery catheter and an operating member in some embodiments.

FIG. 16 is a schematic diagram of an anchoring device released from a delivery catheter in some embodiments.

FIG. 17 is a partial enlarged view of FIG. 16.

FIG. 18 is an assembly diagram of a skirt-side pad, an anchoring device and a delivery catheter in some embodiments.

FIG. 19 is an assembly diagram of a skirt-side pad and an anchoring device in some embodiments.

FIG. 20 is a first schematic diagram of a locking mechanism in a first state in some embodiments.

FIG. 21 is a second schematic diagram of a locking mechanism in a first state in some embodiments.

FIG. 22 is a first schematic diagram of a locking mechanism in a second state in some embodiments.

FIG. 23 is a second schematic diagram of a locking mechanism in a second state in some embodiments.

FIG. 24 is a first schematic diagram of an anchoring device and a target tissue that are fixed together in some embodiments.

FIG. 25 is a partial enlarged view at B in FIG. 24.

FIG. 26 is an assembly diagram of an anchoring device and a second mandrel in some embodiments.

FIG. 27 is a second schematic diagram of an anchoring device and a target tissue that are fixed together in some embodiments.

FIG. 28 is a first schematic diagram of an anchoring device released from a delivery catheter in some embodiments.

FIG. 29 is a second schematic diagram of an anchoring device released from a delivery catheter in some embodiments.

FIG. 30 is a seventh schematic diagram of an anchoring device in some embodiments.

FIG. 31 is an eighth schematic diagram of an anchoring device in some embodiments.

FIG. 32 is a ninth schematic diagram of an anchoring device in some embodiments.

FIG. 33 is a first schematic diagram of an anchoring device anchored to a target tissue in some embodiments.

FIG. 34 is a second schematic diagram of an anchoring device anchored to a target device in some embodiments.

FIG. 35 is a tenth schematic diagram of an anchoring device in some embodiments.

FIG. 36 is an eleventh schematic diagram of an anchoring device in some embodiments.

FIG. 37 is a schematic diagram of a delivery device in some embodiments.

FIG. 38 is a schematic diagram of an anchoring member at a distal end of the anchoring device after being released in some embodiments.

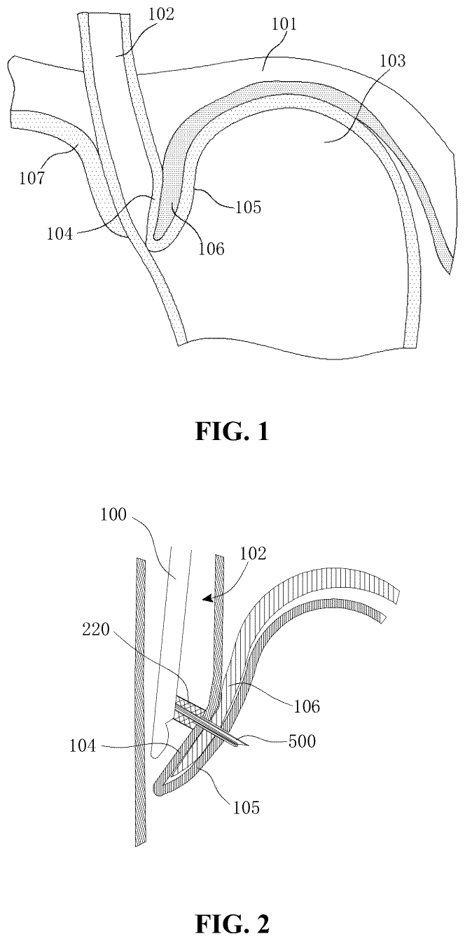

### DESCRIPTION OF EMBODIMENTS

The present application provides a medical apparatus for anchoring two tissue structures. Specifically, the present application discloses an anchoring device and an anchoring apparatus. The anchoring device can be used to secure two or more tissue structures such that the target tissue can restore to be normal. Exemplarily, the anchoring device provided in the present application can be used for a gastrointestinal procedure. Specifically, the anchoring device provided by the present application can be configured to secure a gastric wall, diaphragmatic muscle, and esophageal wall to restore an anti-reflux function at a junction between an esophagus and a stomach.

The term “distal end” used in the present application refers to an end far away from an operator, and the term “proximal end” refers to an end close to the operator.

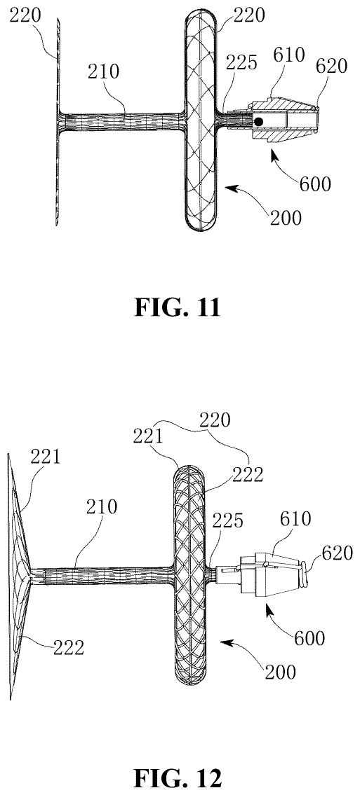

In some implementable embodiments, as shown in FIGS. 6 to 14, the anchoring device 200 provided by the present application includes a coupling member 210 and two anchoring members 220, the two anchoring members 220 are arranged on the coupling member 210 at intervals along a first direction, a dimension of the coupling member 210 in a second direction is a first dimension, the second direction is perpendicular to the first direction. The anchoring member 220 is configured to switch between a first configuration and a second configuration. A dimension of the anchoring member 220 in a direction perpendicular to the first direction is a second dimension when the anchoring member 220 is in the first configuration. A dimension of the anchoring member 220 in the second direction is a third dimension when the anchoring device 200 is in the second configuration, the third dimension is greater than the second dimension and the first dimension.

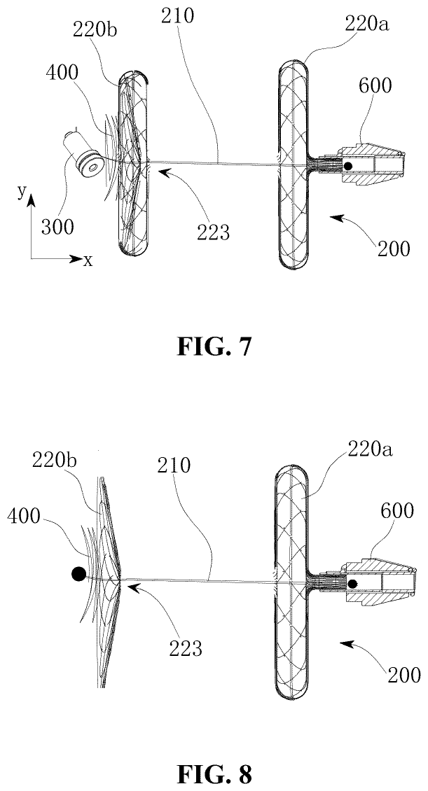

In some embodiments, as shown in FIG. 7, one of the two anchoring members 220 is a first anchoring member 220a and the other one is a second anchoring member 220b. Exemplarily, the first direction may be a direction shown by x-axis in FIG. 7. The second direction may be a direction shown by y-axis in FIG. 7.

In some implementable embodiments, as shown in FIGS. 6 to 14, the anchoring apparatus provided by the present application includes a delivery device 500 and an anchoring device 200. Where, the delivery device 500 includes a delivery catheter 510. The anchoring device 200 is movably arranged within the delivery catheter 510 and is configured to thread out of one end of the delivery catheter 510.

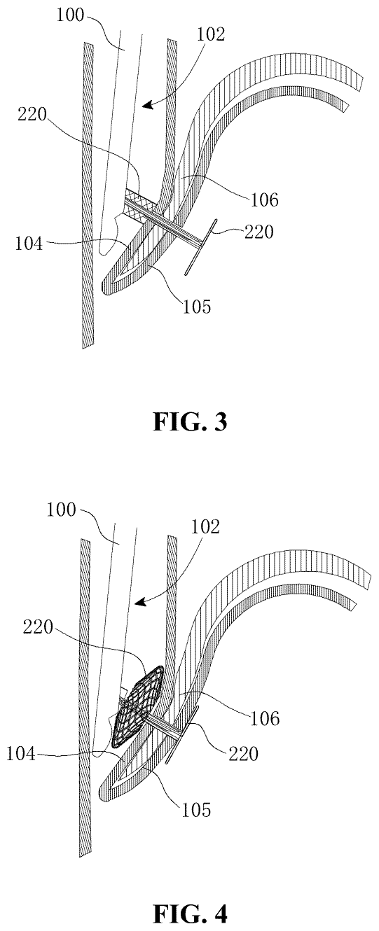

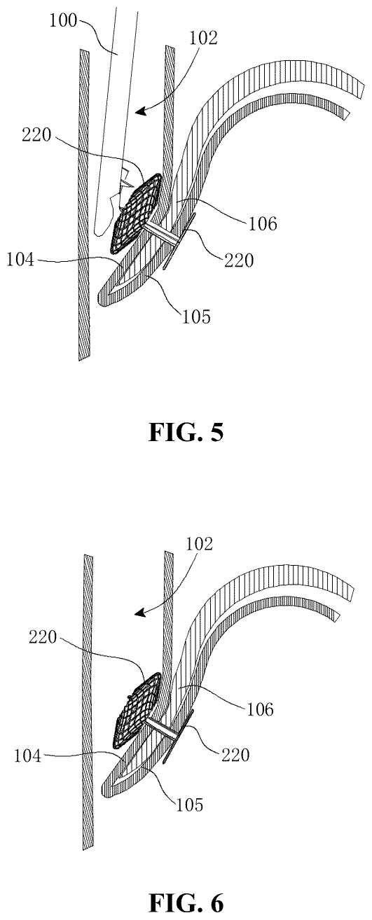

Referring to FIGS. 2 to 5, the anchoring device 200 may be released from one end of the delivery catheter 510 along the delivery catheter 510 such that the anchoring member 220 within the anchoring device 200 may switch between the first configuration and the second configuration.

In some embodiments, the anchoring member 220 is in the first configuration when the anchoring device 200 is positioned within the delivery catheter 510. The anchoring member 220 is in the second configuration when the anchoring device 200 is positioned outside the delivery catheter 510.

In some implementations, when the anchoring device 200 is positioned within the delivery catheter 510, the delivery catheter 510 is configured to constrain the anchoring member 220 to enable the anchoring member 220 to maintain the first configuration. When the anchoring device 200 is positioned outside the delivery catheter 510, the constraint of the delivery catheter 510 on the anchoring member 220 is released and the anchoring member 220 is released to the second configuration.

Referring to FIGS. 2 to 5, during an actual operation, the delivery catheter 510 may extend through a target tissue along a forceps channel of an endoscope 100. Exemplarily, the endoscope 100 is an ultrasound endoscope, which may confirm a location of the target tissue to be anchored through an ultrasound image, such that one end of the delivery catheter 510 is inserted into the location of the target tissue. The anchoring device 200 is disposed within the delivery catheter 510, and is close to one end of the delivery catheter 510 inserted into the body. Exemplarily, one end of the delivery catheter 510 that is inserted into the body is a distal end of the delivery catheter 510, and one end of the delivery catheter 510 that is far away from the body is a proximal end of the delivery catheter 510. In an embodiment, one of two anchoring members 220 of the anchoring device 200 that is far away from the proximal end of the delivery catheter 510 is a distal-end anchoring member, and the other that is close to the proximal end of the delivery catheter 510 is a proximal-end anchoring member.

Referring to FIGS. 2 to 6, in some embodiments, in the process of anchoring a first target tissue and a second target tissue by the anchoring apparatus, the distal end of the delivery catheter 510 is first inserted into the body, so that the delivery catheter 510 penetrates through the first target tissue and the second target tissue to an inner cavity of the second target tissue.

In an embodiment, the delivery catheter 510 and the anchoring device 200 are operated to perform a relative motion until the distal-end anchoring member of the anchoring device 200 is released from the distal end of the delivery catheter 510, so that the distal-end anchoring member may switch to the second configuration from the first configuration.

Further, the delivery catheter 510 moves towards a direction that it is withdrawn from the body, so that at least part of the distal-end anchoring member may abut against an internal face of the second target tissue, so as to realize that the distal-end anchoring member is adjoined to the internal face of the second target tissue.

Further, the delivery catheter 510 continues to move towards the direction of withdrawing from the body, so that the proximal-end anchoring member of the anchoring device 200 may move to the inner cavity of the first target tissue. Then, the delivery catheter 510 and the anchoring device 200 are operated to perform a relative motion until the proximal-end anchoring member of the anchoring device 200 is released from the distal end of the delivery catheter 510, so that the proximal-end anchoring member can switch to the second configuration from the first configuration, and the proximal-end anchoring member may abut against the internal face of the first target tissue to realize that the proximal-end anchoring member is adjoined to the internal face of the first target tissue.

In some embodiments, an outside diameter of the delivery catheter 510 is a fourth dimension, and the third dimension is larger than the fourth dimension. In this way, after the delivery catheter 510 penetrates through the target tissue, the distal-end anchoring member 220 is released, so that at least part of the distal-end anchoring member 220 can protrude from an outer peripheral wall of the delivery catheter 510 after it is deployed, which is beneficial to prevent the anchoring member 220 from retreating to the proximal end along a through-hole disposed on the target tissue with the delivery catheter 510, and ensure that the distal-end anchoring member 220 may abut against one side of the target tissue far away from the proximal end of the delivery catheter 510. Similarly, the proximal-end anchoring member 220 switches to the second configuration after being released, which is beneficial to ensure that the proximal-end anchoring member 220 may abut against one side of the target tissue close to the proximal end of the delivery catheter 510 after it is deployed. Therefore, the above embodiment is beneficial to prevent the anchoring device 200 and the target tissue from being loose and enhance the reliability of connection between the anchoring device 200 and the target tissue.

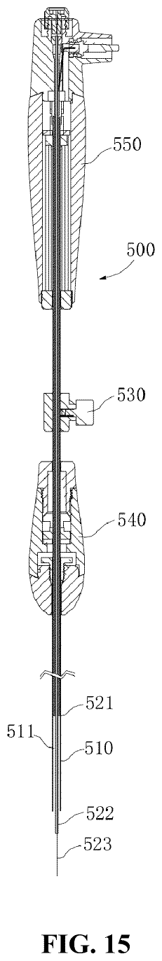

In some embodiments, referring to FIGS. 15 to 17, the delivery device 500 further includes a second mandrel 520, and at least part of the second mandrel 520 is positioned within the delivery catheter 510, and the second mandrel 520 is configured to be movable relative to the delivery catheter 510. In an embodiment, a first end of the second mandrel 520 is detachably coupled with the anchoring device 200. Exemplarily, at least part of a second end of the second mandrel 520 extends from one end of the delivery catheter 510 far away from the anchoring device 200, that is, at least part of the second end of the second mandrel 520 extends from the proximal end of the delivery catheter 510, so as to facilitate to operate the delivery catheter 510 to move relative to the second mandrel 520, thereby driving the anchoring device 200 to move relative to the delivery catheter 510.

Exemplarily, the proximal end of the delivery catheter 510 is operated by an operator, so that the delivery catheter 510 may move along the second mandrel 520 to the proximal end of the second mandrel 520, then the anchoring device 200 moves relative to the delivery catheter 510, thereby realizing that the anchoring member 220 in the anchoring device 200 can be released from the distal end of the delivery catheter 510 and switch to the second configuration from the first configuration.

In other embodiments, the proximal end of the second mandrel 520 may also be operated by the operator, so that the second mandrel 520 may move along the delivery catheter 510 to the distal end of the delivery catheter 510 and then the anchoring device 200 is driven to move to the distal end of the delivery catheter 510 relative to the delivery catheter 510 until the anchoring member is released out of the delivery catheter 510 from the distal end of the delivery catheter 510.

In some embodiments, the distal-end anchoring member 220 in the anchoring device 200 can be released by pushing the second mandrel 520, and then the delivery catheter 510 can be operated to cause the delivery catheter 510 to retract back towards the proximal end relative to the second mandrel 520, thereby realizing that the proximal-end anchoring member 220 is released.

In some embodiments, the delivery device 500 further includes a limiting member 530 arranged on the second mandrel 520, and the limiting member 530 is positioned at one side of the delivery catheter 510 far away from the anchoring device 200. The limiting member 530 is configured to switch between a first state and a second state, and can abut against one side of the delivery catheter 510 far away from the anchoring device 200 and limit position. The limiting member 530 is in limiting-fit with the second mandrel 520 in an extension direction of the second mandrel 520 when the limiting member 530 is in the first state. When the limiting member 530 is in the second state, the limiting member 530 is in slide-fit with the second mandrel 520 and the limiting member 530 slides along the extension direction of the second mandrel 520.

In a process of securing a tissue structure by an anchoring apparatus, the limiting member 530 can be arranged at a preset location of the second mandrel 520 to regulate a relative movable displacement of the delivery catheter 510 and the second mandrel 520. Exemplarily, prior to releasing of the distal-end anchoring member 220, a distance between the limiting member 530 and the proximal end of the delivery catheter 510 may be set as required. In a process of releasing the distal-end anchoring member 220, it is only necessary to retract the delivery catheter 510 back to the proximal end until the proximal end of the delivery catheter 510 abuts against the limiting member 530, thereby ensuring that only the distal-end anchoring member 220 is released, and then preventing the two anchoring members 220 from being released together. Further, the limiting member 530 can be switched to the second state when the proximal-end anchoring member 220 needs to be released, so that the delivery catheter 510 can continue to retract back towards the proximal end relative to the second mandrel 520, thus realizing that the proximal-end anchoring member 220 is released.

In some embodiments, the limiting member 530 includes a sliding portion and a locking portion. The sliding portion is provided with a sliding hole, and the sliding portion is sleeved on the second mandrel 520 through the sliding hole, so that the sliding portion can move along an extension direction of the second mandrel 520. In an embodiment, the limiting member 530 is also provided with a threaded hole penetrating through a hole wall of the sliding hole and communicating with the sliding hole. In an embodiment, the locking portion has a screw portion, and the screw portion of the locking portion is in screw-thread fit with the threaded hole, so as to realize the limiting fit and releasing of the limiting-fit between the limiting member 530 and the second mandrel 520 by twisting the locking portion.

In some embodiments, a length of the screw portion of the locking portion in its axis direction is larger than a dimension of the threaded hole in its axis direction. Exemplarily, when the limiting member 530 is in the first state, the screw portion is trapped on an inside wall of the sliding hole so that the sliding portion can slide along the second mandrel 520. When the limiting member 530 is in the second state, at least part of the screw portion protrudes from the inside wall of the sliding hole, so that the screw portion can abut against an outer lateral wall of the second mandrel 520 and is in limiting-fit with the second mandrel 520. Further, the limiting member 530 can switch between the first state and the second state by an operation of twisting the locking portion.

In other embodiments, an anti-skid pad is disposed in the threaded hole, and one end of the screw portion abuts against the anti-skid pad, the anti-skid pad abuts against an outer surface of the second mandrel 520 by twisting of the screw portion, thereby realizing the limiting-fit between the limiting member 530 and the second mandrel 520.

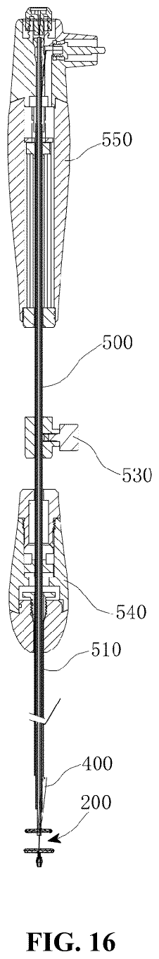

In some embodiments, as shown in FIGS. 15 and 16, the delivery device 500 further includes a first operating member 540 and a second operating member 550. Exemplarily, the first operating member 540 is coupled with the proximal end of the delivery catheter 510, so that the operator may hold the first operating member 540 to operate the delivery catheter 510 to move along its extension direction.

In an embodiment, the second operating member 550 is fixedly coupled with a part of the second mandrel 520 that runs out of the first operating member 540. Exemplarily, the second operating member 550 is fixedly coupled with the proximal end of the second mandrel 520, so as to drive the second mandrel 520 to be pushed toward the distal end or be retracted back towards the proximal end by operating the second operating member 550. Exemplarily, in a process of anchoring the target device by the anchoring device 200, the delivery catheter 510 may be pulled back by making the first operating member 540 withdraw along the second mandrel 520, thus releasing the anchoring device 200.

In some embodiments, a part of the second mandrel 520 that runs out of the first operating member 540 is provided with a guide tube. One end of the guide tube is fixedly coupled with the second operating member 550, and at least part of the other end of the guide tube is inserted into the first operating member 540 and is in slide-fit with the first operating member 540. Exemplarily, the guide tube is made of a hard material and provides a guide for the second mandrel 520 moving to the distal end relative to the first operating member 540, so as to avoid the part of the second mandrel 520 that runs out of the first operating member 540 being bent under force. Specifically, the guide tube may be made of, but not limited to, a stainless steel.

Exemplarily, the limiting member 530 is arranged on the guide tube, and the limiting member 530 is configured to switch between the first state and the second state. The limiting member 530 is in limiting-fit with the first operating member 540 in an extension direction of the guide tube when the limiting member 530 is in the first state. When the limiting member 530 is in the second state, the limiting member 530 is in slide-fit with the guide tube and the limiting member 530 slides along the extension direction of the guide tube.

In some embodiments, a first end of the second mandrel 520 is detachably coupled with the anchoring device 200, so that the second mandrel 520 may be detached from the anchoring device 200.

In some embodiments, the second mandrel 520 is coupled with the anchoring device 200 via a thread-fitting manner, so that the second mandrel 520 may be detached from the anchoring device 200 by rotating the second mandrel 520.

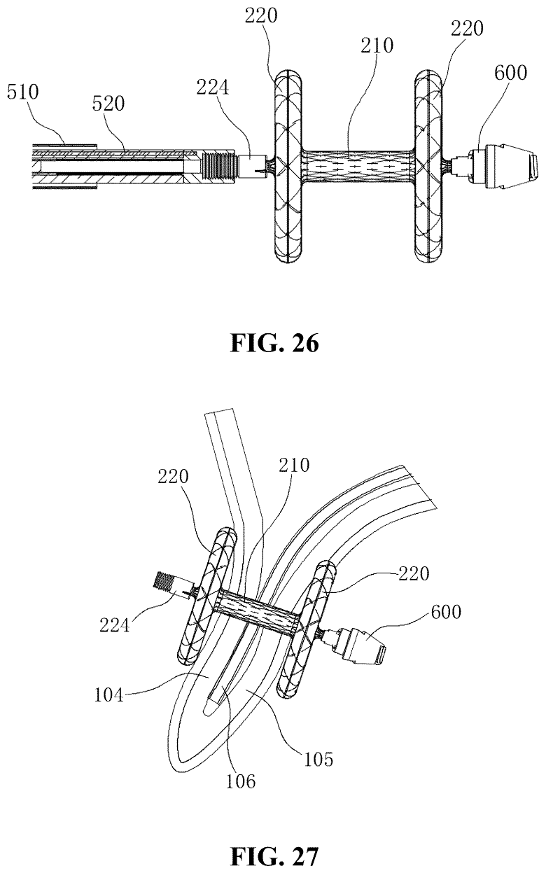

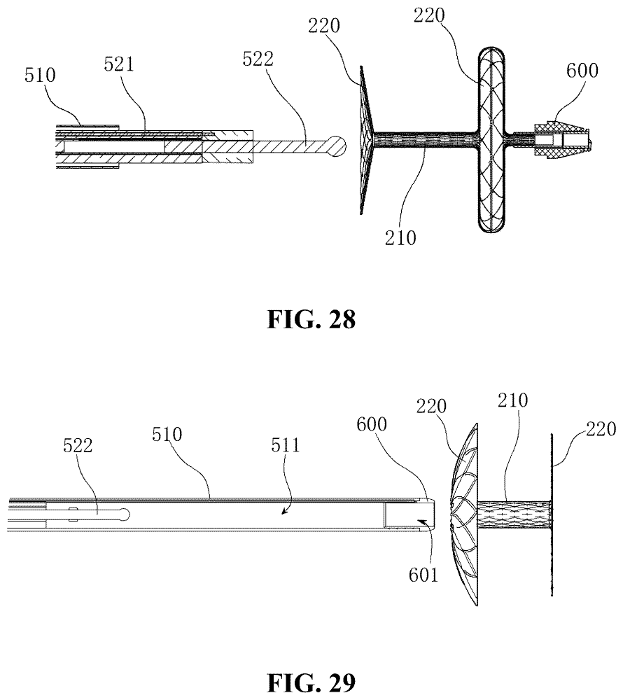

In some embodiments, as shown in FIGS. 26 and 27, the proximal-end anchoring member 220 further includes a coupling portion 224. Exemplarily, one of the coupling portion 224 and the distal end of the second mandrel 520 has an external thread and the other has an internal thread, so that the coupling portion 224 may be coupled with the distal end of the second mandrel 520 via a thread-fitting.

In some embodiments, an assembly gap is disposed between the second mandrel 520 and the delivery catheter 510. The anchoring member 220 is positioned between the second mandrel 520 and the delivery catheter 510 when the anchoring device 200 is positioned within the delivery catheter 510. A space for accommodating the anchoring member 220 may be formed by constraining effect of the second mandrel 520 and the delivery catheter 510 in this embodiment. In some embodiments, the second mandrel 520 and the delivery catheter 510 may be used to constrain the anchoring member 220 to maintain the first configuration. In addition, the second mandrel 520 may further provide support for the anchoring device 200.

In some embodiments, a dimension of the anchoring member 220 in the second direction decreases in a process of the anchoring member 220 switching to the first configuration from the second configuration, then the dimension of the anchoring member 220 in the first direction increases. In the above embodiment, the anchoring member 220 is sleeved on the distal end of the second mandrel 520 when the anchoring member 220 is in the first configuration, and then the second mandrel 520 may be used to provide support for the delivery catheter 510 and the anchoring device 200, which is beneficial to ensure the strength a part of the delivery catheter 510 close to the distal end, so that the delivery catheter 510 penetrates through the target tissue.

In some embodiments, an outer peripheral wall of the second mandrel 520 is provided with a butting portion. In an implementation, the butting portion protrudes from the outer peripheral wall of the second mandrel 520. At least part of the proximal end of the anchoring device 200 abuts against the butting portion when the anchoring device 200 is positioned between the second mandrel 520 and the delivery catheter 510. This is beneficial to prevent the anchoring device 200 from moving towards the proximal end along the second mandrel 520 under a friction of the delivery catheter 510.

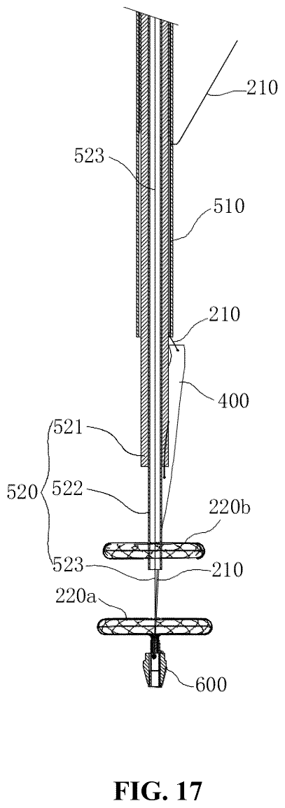

In other embodiments, as shown in FIGS. 16 and 17, the second mandrel 520 includes a second catheter 521 and a third catheter 522 arranged within the second catheter 521, and at least part of one end of the third catheter 522 close to the anchoring device 200 runs out of the second catheter 521; one end of the second catheter 521 close to the anchoring device 200 abuts against the anchoring device 200.

In the above embodiment, the distal end of the second catheter 521 can be used to abut against the anchoring device 200 to prevent the anchoring device 200 from moving towards the proximal end along the second mandrel 520 under a friction of the delivery catheter 510.

Referring to FIGS. 16 and 17, in some embodiments, the second mandrel 520 further includes a support wire 523 movably arranged in the third catheter 522. At least part of the support wire 523 runs out of one end of the third catheter 522 close to the anchoring device 200 when the anchoring device 200 is positioned in the delivery catheter 510.

In an implementation, the support wire 523 may be made of, but not limited to, a metal.

In the above embodiment, the support wire 523 may provide support for the second mandrel 520 and the delivery catheter 510, which is beneficial to improve the strength of the second mandrel 520 and the delivery catheter 510.

In some embodiments, the delivery device 500 of the anchoring apparatus further includes a piercing head 600, which is arranged at an end portion of the delivery catheter 510 and is configured to penetrate through a tissue structure of a surgical object.

In other embodiments, the piercing head 600 is arranged at an end portion of the second mandrel 520 or of the anchoring device 200.

During an operation, the target tissue may be pierced by the piercing head 600 to facilitate the delivery catheter 510 to penetrate through the target tissue.

In some embodiments, the piercing head 600 may be a hot piercing head or a cold piercing head.

In some embodiments, the piercing head 600 includes a thermal-insulation member 610 and a conductor 620, the conductor 620 is arranged on the thermal-insulation member 610. In some embodiments, during a process of the anchoring device 200 implanting into a target tissue, the conductor 620 is energized to cut the target tissue. In some embodiments, the delivery device 500 may be externally connected with a high-frequency generator, and high-frequency current is transmitted to a front end of the piercing head 600 for thermal puncture. Exemplarily, the thermal-insulation member 610 may be made of a ceramic material. The conductor 620 is made of a metal conductive material. Exemplarily, the material of conductor 620 may be a stainless steel. In an embodiment, a conductive connecting-line is provided in the second mandrel 520. One end of the conductive connecting-line is connected with the high-frequency generator, and the other end may abut against the conductor 620 to connect the high-frequency generator with the conductor 620.

In some embodiments, the anchoring apparatus is taken as an example to restore an anti-reflux function at a junction between the esophagus 102 and the gastric fundus 103. The piercing head 600 is positioned at the distal end of the anchoring device 200, and is configured to pass through a space formed by the esophageal wall 104, the gastric fundic wall 105, and the left limb of right crus of diaphragm 106, so that the anchoring device 200 can pass through the esophageal wall 104, the gastric fundic wall 105, and the left limb of right crus of diaphragm 106 from the esophagus 102 to the gastric fundus 103. Referring to FIG. 12, the piercing head 600 includes the thermal-insulation member 610 and the conductor 620. In an implementation, the anchoring apparatus further includes a conducting wire. In an implementation the conducting wire may extend along the delivery catheter 510 and/or the second mandrel 520 to the anchoring device 200 and be coupled with the piercing head 600. Exemplarily, the conductor 620 is coupled with the conducting wire that extends to the anchoring device 200, so as to supply power by the conducting wire to the piercing head 600 for cutting the target tissue. In an example, the conducting wire is directly fixedly connected to the piercing head 600 without passing through the anchoring device 220, which greatly reduces resistance and achieves good thermal puncture effect.

In some embodiments, the coupling member 210 can be fixedly coupled with one end of the piercing head 600 close to the anchoring device 200 when the piercing head 600 is arranged at the distal end of the anchoring device 200.

Referring to FIG. 10, in some embodiments, the piercing head 600 is arranged in the anchoring device 200 and is close to the anchoring member 220 at the distal end of the anchoring device 200. The piercing head 600 further includes a coupling buckle 630, a first end of the coupling buckle 630 is coupled with the thermal-insulation member 610, a second end of the coupling buckle 630 is coupled with the coupling member 210. In an implementation the coupling buckle 630 can be made of a heat-resistant material. Exemplarily, the coupling buckle 630 can be, but not limited to, metal and composite materials.

In the above embodiments, the coupling buckle 630 can increase a distance between the coupling member 210 and both the conductor 620 and the thermal-insulation member 610, which is beneficial to prevent the coupling member 210 from being broken or loosen after being heated.

In an implementation, the coupling buckle 630 may be of a “U” type structure. Exemplarily, the coupling buckle 630 may be a metal wire, and the coupling buckle 630 is bent to form a “U” structure. In an embodiment, both ends of the metal wire are welded with the conductor 620. The coupling member 210 is coupled with a bending part of the metal wire. In other embodiments, an end portion of the coupling buckle 630 may also be riveted in the conductor 620.

In other embodiments, the distal end of the anchoring device 200 is further provided with an extension portion 225. Exemplarily, the extension portion 225 extends from the distal end of the anchoring device 200 to a direction far away from the proximal end of the anchoring device 200. In an implementation, a first end of the extension portion 225 is coupled to the piercing head 600, a second end of the extension portion 225 is coupled to the anchoring member 220 at the distal end of the anchoring device 200. In some embodiments, the extension portion 225 may include the same wire mesh material as the anchoring member 220 at the distal end or the anchoring member 220 at the proximal end of the anchoring device 200, and is configured to provide a sufficient space between the piercing head 600 and the distal-end anchoring member 220 to prevent the distal-end anchoring member 220 from being damaged by the heat generated by the conductor 620 and the thermal-insulation member 610.

In some embodiments, during a process of implanting the anchoring device 200 into the body, the anchoring device 200 is inserted into the delivery catheter 510, and then the delivery catheter 510 is inserted into the body. The piercing head 600 is activated upon the distal end of the delivery catheter 510 reaching the target tissue. Exemplarily, the conductor 620 in the piercing head 600 is coupled with the high-frequency generator to form a cutting effect through high-frequency electricity, and penetrates into the target tissue to be anchored.

Exemplarily, under a condition that the anchoring apparatus is configured to reshape the morphology of the junction between esophagus 102 and gastric fundus 103 to restore the anti-reflux function at the junction between esophagus 102 and gastric fundus 103, the high frequency electricity is provided for the piercing head 600 through the conducting wire. In an implementation, the proximal end of the delivery catheter 510 pushes the piercing head 600 towards the distal end to cause the high frequency current to pass through the piercing head 600, thereby forming a high frequency cutting puncture effect. Exemplarily, the piercing head 600 passes through the esophageal wall 104, the left limb of right crus of diaphragm 106, and the gastric fundic wall 105 in turn, and then penetrates through the esophageal wall 104, the left limb of right crus of diaphragm 106, and the gastric fundic wall 105. The delivery catheter 510 is retracted back proximally to expose the distal-end anchoring member 220 of the anchoring device 200, that is, the gastric anchoring member is released to cause the distal-end anchoring member 220 in the anchoring device 200 to expand within the gastric fundus 103 and switch to the second configuration. The anchoring device 200 is further retracted back towards the proximal end of the delivery catheter 510 until the distal-end anchoring member 220 of the anchoring device 200 contacts the gastric fundic wall 105. The delivery catheter 510 is further retracted back towards the proximal end of the delivery device 500 to expose the proximal-end anchoring member 220 of the anchoring device 200, that is, the esophageal anchoring member is released to cause the proximal-end anchoring member 220 of the anchoring device 200 to expand within the esophagus 102 and switch to the second configuration, so that the proximal-end anchoring member 220 of the anchoring device 200 contacts the esophageal wall 104.

In some embodiments, when the distal-end anchoring member 220 of the anchoring device 200 is in contact with the gastric fundic wall 105, the coupling member 210 is configured to extend as the delivery device 500 moves towards the proximal end (e.g., when the distal-end anchoring member 220 of the anchoring device 200 is in contact with the gastric fundic wall 105, the proximal-end anchoring member of the anchoring device 200 is driven to move toward the proximal end of the delivery device 500 during the movement of the delivery device 500 towards the proximal end, thereby tensioning the coupling member 210 to make the coupling member 210 under tension, and then causing the coupling member 210 to lengthen). When the proximal-end anchoring member 220 of the anchoring device 200 is released from the delivery catheter 510 and switched to the second configuration, tension within the coupling member 210 is released, and the coupling member 210 contracts. Accordingly, the length of the coupling member 210 is adjustable based on the distance from the esophageal wall 104 to the gastric fundic wall 105.

In some embodiments, the coupling member 210 will not contract to its resting state, but remain under tension. This tension causes the proximal-end anchoring member 220 and the distal-end anchoring member 220 of the anchoring device 200 to contact the esophageal wall 104 and the gastric fundic wall 105, respectively, such that the esophageal wall 104, the left limb of right crus of diaphragm 106, and the gastric fundic wall 105 are held together with a compressive force. The esophageal wall 104, the left limb of right crus of diaphragm 106, and the gastric fundic wall 105 are hold together with a compressive force. The esophageal wall 104, the left limb of right crus of diaphragm 106, and the gastric fundic wall 105 are squeezed together by long-time compressive force, which can form fusion. Once the esophageal wall 104, the left limb of right crus of diaphragm 106, and the gastric fundic wall 105 form fusion, even if the anchoring device 200 is removed, the esophageal wall 104, the left limb of right crus of diaphragm 106, and the gastric fundic wall 105 are still secured together to maintain the anti-reflux structure and realize the anti-reflux function.

In some embodiments, the coupling member 210 is flexibly coupled with the anchoring member 220. This is beneficial for an included angle between the coupling member 210 and the anchoring member 220 to be able to fit the morphology of the target tissue.

In some embodiments, the two anchoring members 220 in the anchoring device 200 and the coupling member 210 may be coupled through a flexible coupling line, so that an included angle between the two anchoring members 220 and the coupling member 210 may conform to the morphology of the secured target tissue, which is beneficial to improve a compliance of a surface fitting of the anchoring member 220 and the target tissue, and enhance a reliability of combination of the anchoring device 200 and the target tissue.

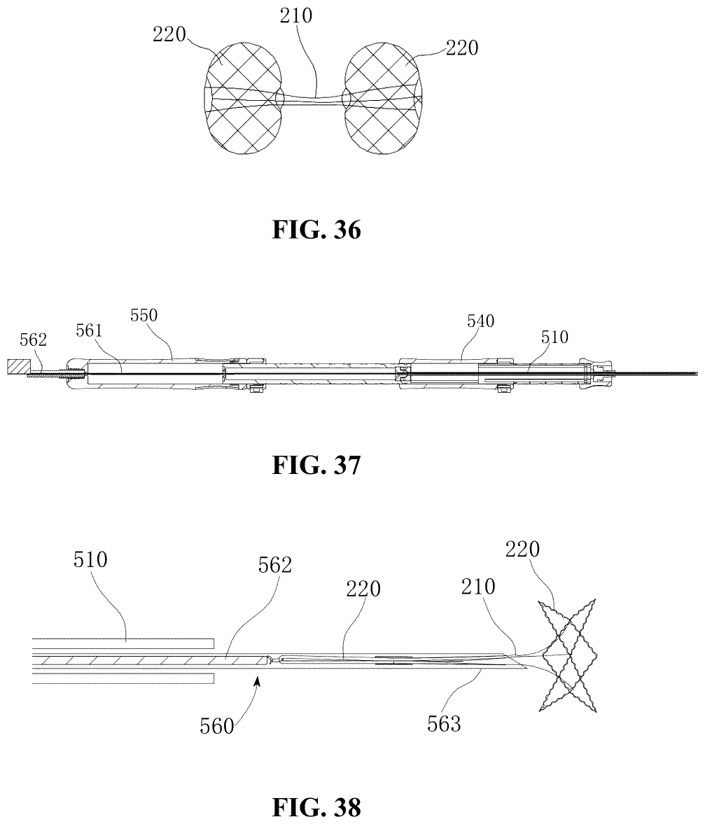

Referring to FIG. 35 or FIG. 36, according to some embodiments, the coupling member 210 is a flexible coupling line. In this way, the coupling member 210 and the anchoring member 220 may not only realize a flexible connection, but also render the coupling member 210 to adapt to the target tissue structure and a shape of a piercing hole of the target tissue for installing the anchoring device, so as to avoid the piercing hole of the target tissue being stretched by an action of the coupling member 210. In a case that the anchoring device is used to reshape the morphology of the junction between the esophagus 102 and the gastric fundus 103, the anchoring device 200 provided by this embodiment is beneficial to prevent the food in the stomach from leaking out along the piercing hole.

In some embodiments, prior to the gastric anchoring member abutting the gastric fundic wall 105, an included angle between a part of the gastric fundic wall 105 for abutting the gastric anchoring member and the coupling member 210 is a first gastric angle. After the gastric anchoring member abuts the gastric fundic wall 105, an included angle between a part of the gastric fundic wall 105 for abutting the gastric anchoring member and the coupling member 210 is a second gastric angle. In an embodiment, the first gastric angle is not equal to the second gastric angle, that is, the morphology of the gastric fundic wall 105 adjacent to the gastric anchoring member can be adjusted through the gastric anchoring member.

In some embodiments, prior to the esophageal anchoring member abutting the esophageal wall 104, an included angle between a part of the esophageal wall 104 for abutting the esophageal anchoring member and the coupling member 210 is a first esophageal angle. After the esophageal anchoring member abuts the esophageal wall 104, an included angle between a part of the esophageal wall 104 for abutting the esophageal anchoring member and the coupling member 210 is a second esophageal angle. In an embodiment, the first esophageal angle is not equal to the second esophageal angle, that is, the morphology of the esophageal wall 104 adjacent to esophageal anchoring member can be adjusted through the esophageal anchoring member.

In some embodiments, as shown in FIG. 29, the part of the at least one anchoring member 220 in the anchoring device 200 that contacts with a surface of the anchored tissue has the largest diameter, and the diameter of the anchoring member 220 decreases as it moves away from the surface of the anchored tissue. Exemplarily, the anchoring member 220 is provided as an umbrella shape.

In some embodiments, the delivery catheter 510 defines a channel 511 for the second mandrel 520 and/or the anchoring device 200 to move. Exemplarily, the delivery catheter 510 can be formed of any material that is suitable for its intended purpose of accessing the interior of a body. In an implementation, the delivery catheter 510 can be made of, but not limited to, polyethylene, Pebax (polyether block polyamide), PTFE (Poly Tetra Fluoroethylene) or composite materials, such as a metal mesh pipe, etc.

The piercing head 600 is positioned at the distal end of the delivery catheter 510 and is configured to increase temperature in response to an electric current being provided to the piercing head 600 via the conducting wire extending along the delivery catheter 510. In some embodiments, one or more wires are embedded in the delivery catheter 510 and extend along the delivery catheter 510. In some embodiments, one end of the conducting wire for coupling with the piercing head 600 runs out of the proximal end of the delivery catheter 510 and is connected to an electronic power source; and the other end of the conducting wire is coupled with the piercing head 600 at the distal end of the delivery catheter 510 to provide electric current for the piercing head 600.

In some embodiments, the second mandrel 520 is configured to fit and move along a channel 511 defined by the delivery catheter 510. Exemplarily, the second mandrel 520 can provide support for the delivery catheter 510 and ensure that the delivery catheter 510 can maintain its own shape in a process of piercing the tissue in the body.

In some embodiments, the second mandrel 520 is generally formed from one or more materials that are stiffer and/or stronger than the delivery catheter 510. In an implementation, the second mandrel 520 may be also configured to slide within the channel 511 defined by the delivery catheter 510 such that the second mandrel 520 can push the distal-end anchoring member 220 and the proximal-end anchoring member 220 of the anchoring device 200 out of the distal end of the delivery catheter 510, thereby realizing that the anchoring device 200 is secured to the target tissue.

In some embodiments, the piercing head 600 may also be a needle-shaped piercing head.

Referring to FIGS. 29 and 38, in some embodiments, the piercing head 600 has a first avoidance hole 601 that penetrates through the piercing head 600, and the anchoring device 200 is configured to extend out of the first avoidance hole 601.

In some embodiments, as shown in FIG. 29, the piercing head 600 is arranged at one end of the delivery catheter 510 close to the anchoring device 200, the first avoidance hole 601 is communicated with the delivery catheter 510. In this way, the anchoring device 200 may move from a proximal end of the piercing head 600 to a distal end of the piercing head 600 along the first avoidance hole 601, and extend out from the distal end of the piercing head 600.

In the above embodiment, the piercing head 600 is directly arranged at the distal end of the delivery catheter 510. During an operation process, by operating the proximal end of the delivery catheter 510, the operator transfers a force to the piercing head 600 using the delivery catheter 510, so that the piercing head 600 can penetrate through the target tissue.

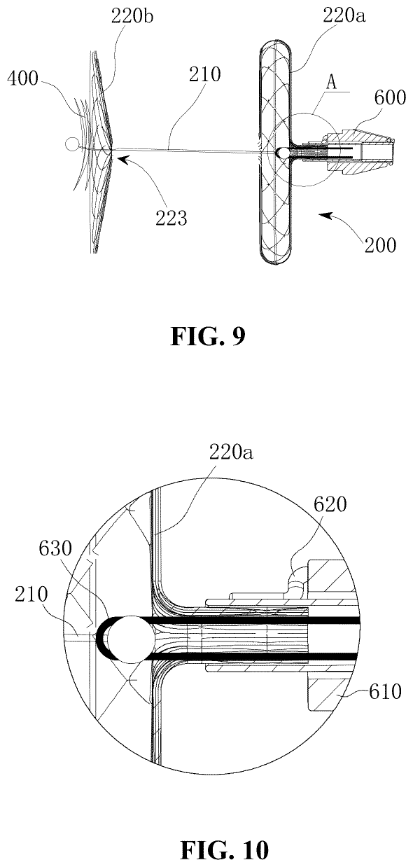

In some embodiments, the piercing head 600 is detachably coupled with one end of the anchoring device 200 far away from the second mandrel 520. Exemplarily, the piercing head 600 is coupled with the distal end of the anchoring device 200. In some embodiments, as shown in FIGS. 9 to 11, the piercing head 600 is coupled with the proximal-end anchoring member 220 of the anchoring device 200 far away from the delivery catheter 510.

In the above embodiment, the delivery catheter 510 and the second mandrel 520 may move synchronously during piercing of the piercing head 600. Specifically, the delivery catheter 510 can provide support for the second mandrel 520, which is beneficial to prevent the second mandrel 520 from being bent. The second mandrel 520 pushes the anchoring device 200 and drives the piercing head 600 to puncture the target tissue by the anchoring device 200.

In some embodiments, the piercing head 600 can be fixedly coupled with the distal end of the anchoring device 200 and remained in the body with the anchoring device 200.

In other embodiments, the piercing head 600 can be detachably coupled with the distal end of the anchoring device 200, and then the piercing head 600 can be detached from the anchoring device 200 after the puncture is finished.

In some embodiments, the piercing head 600 is coupled with one end of the anchoring device 200 far away from the second mandrel 520 via a thread connection. In an implementation, the piercing head 600 can be detached from the anchoring device 200 by a rotation manner.

In an example, the piercing head 600 may be fixedly coupled (e.g. welded/fused) or detachably coupled with the distal end (i.e. the first end) of the second mandrel 520, e.g. threaded connection, snap connection, or sliding connection. In this case, after the release of the proximal-end and distal-end anchoring members is completed, the piercing head 600 can be withdrawn from the anchoring tissue along an axis passing through the through-holes located at the center of the two anchoring members along with the delivery catheter 510.

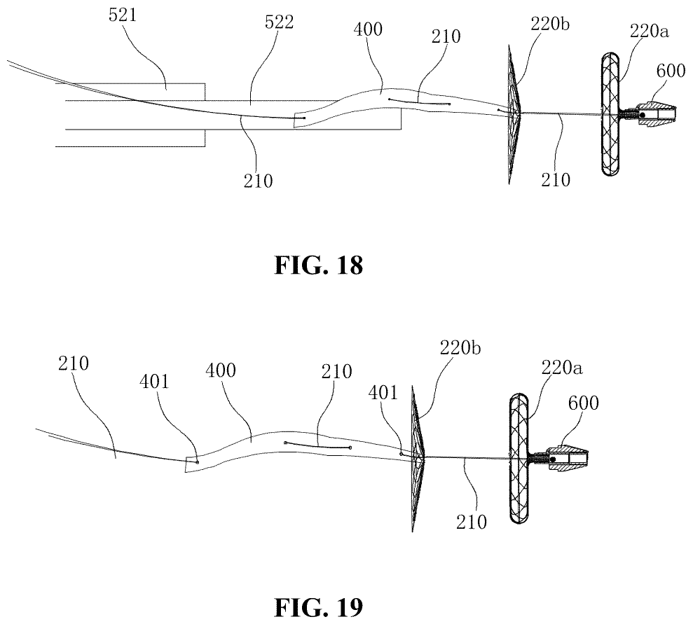

In an example, the piercing head 600 is tapered at its two ends. For example, as shown in FIGS. 18-19, the proximal end of the piercing head 600 is stepped and its distal end is in a shape of truncated cone. Furthermore, the proximal end of the piercing head 600 may be in a shape of shuttle with a smooth transition surface, thereby preventing the piercing head 600 from getting stuck by an anti-reflux valve located at the through-hole in the center of the anchoring member, the anchoring member, or the wire mesh that forms the anchoring member during retraction of the piercing head, and further reducing the resistance encountered during retraction.

In an example, a through-hole is provided at the center of the anchoring member 220 for the piercing head to pass through, the through-hole having a diameter of approximately 4 mm (as shown in FIG. 14). The through hole of each anchoring member 220 may be equipped with a matching anti-reflux valve, which may be made of silicone and has a gap for the piercing head to pass through. The size of the gap is designed to allow the piercing head to pass through without causing backflow.

In instances where using thermal puncture to create a channel for the penetration of delivery catheter 510 is not desirable, a channel for the penetration of delivery catheter 510 may be created by cutting the target tissue. Exemplarily, in a process of a gastrointestinal procedure, a space or channel for the penetration of delivery catheter 510 can be created by cutting the tissues of the esophageal wall 104, the left limb of right crus of diagram 106, and the gastric fundic wall 105.

In some embodiments, the delivery device 500 includes a third mandrel 560, and the anchoring device 200 can be pushed to move within the delivery catheter 510 by the third mandrel 560. Exemplarily, the third mandrel 560 includes a fourth catheter 561, a booster 562, and a puncture needle 563. A first end of the puncture needle 563 is a tip, and a target area of the target tissue can be cut and penetrated by the tip of the puncture needle 563. The target area is an area in the target tissue that needs to be anchored by the anchoring device 200. A second end of the puncture needle 563 is coupled with the fourth catheter 561. The puncture needle 563 has a needle hole that penetrates from the first end of the puncture needle 563 to the second end of the puncture needle 563 and is communicated with the fourth catheter 561.

The anchoring device 200 and the booster 562 are movably arranged in the fourth catheter 561 or the puncture needle 563. At least part of the booster 562 is positioned in the fourth catheter 561 or puncture needle 563, and the booster 562 is configured to be movable relative to the fourth catheter 561 and the puncture needle 563. The booster 562 abuts the anchoring device 200, and is configured to push the anchoring device 200 out of the puncture needle 563. Exemplarily, at least part of a proximal end of the booster 562 runs out of the proximal end of the fourth catheter 561, and then the booster 562 can push the anchoring device 200 out of the distal end of the puncture needle 563 and release it by operating the proximal end of the booster 562.

Exemplarily, when the delivery device 500 is a piercing device in FIG. 37, after the delivery device 500 is delivered to a target location via a forceps channel of an endoscopic, the delivery catheter 510 abuts a puncture location of the target tissue, and then the fourth catheter 561 drives the puncture needle 563 at a distal end of the fourth catheter 561 to cut the target area of the target tissue by operating the third mandrel 560, so that the puncture needle 563 can penetrate through the target tissue. Then the booster 562 is used to push the anchoring device 200 in the fourth catheter 561 to release the anchoring member 220 at the distal end of the anchoring device 200. In an implementation, the delivery device 500 is withdrawn in a direction of withdrawing the delivery device 500 from the body until the proximal-end anchoring member 220 of the anchoring device 200 moves to one side of the target tissue close to the proximal end of the delivery device 500. Finally, the anchoring member 220 at the proximal end of the anchoring device 200 passes though and is released from the distal end of the puncture needle 563 via the booster 562.

In some embodiments, the anchoring member 220 includes a wire portion 221, the wire portion 221 is configured to switch between a compression state and a deployed state. The anchoring member 220 is in the first configuration when the wire portion 221 is in the compression state. The anchoring member 220 is in the second configuration when the wire portion 221 is in the deployed state.

In the above embodiment, the anchoring member 220 can reduce a dimension of the anchoring member 220 in the second direction by compression and increase the dimension of the anchoring member 220 in the second direction by deploying the anchoring member 220. Exemplarily, the anchoring member 220 can switch to the compression state by stretching the anchoring member 220 in the first direction.

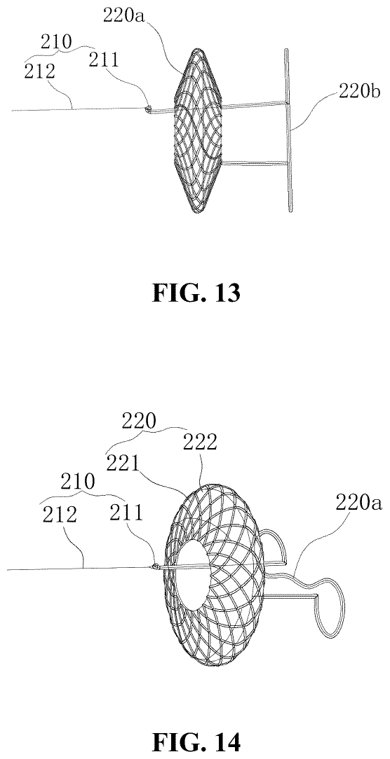

Referring to FIGS. 7 to 14 and 30, in some embodiments, the wire portion 221 forms a foldable mesh structure. As shown in FIGS. 13 and 14, the wire portion 221 can be configured as a wire mesh structure to form an anchoring member 220. Specifically, a size of mesh in anchoring member 220 can be set as required.

In some embodiments, as shown in FIGS. 13 and 14, the wire portion 221 can be constructed into a disc-shaped mesh structure, so that the anchoring member 220 can be compressed or deployed.

In some embodiments, a cross-section shape of the anchoring member 220 in a direction perpendicular to the first direction may be a circular, annular, oval, polygonal, etc., when the anchoring member 220 is in the deployed state. Of course, in other embodiments, the cross-section shape of the anchoring member 220 in the direction perpendicular to the first direction may also be a shape formed by splicing multiple rings, circles and/or polygons. Thus, this embodiment does not limit the specific shape of the anchoring member 220 when it is in the deployed state.

Exemplarily, the wire portion 221 is configured as an annular disc-shaped structure. The anchoring member 220 may be shaped as a disc such that the anchoring member 220 has a rectangular cross-section, as shown in FIGS. 7 to 12. Exemplarily, the anchoring member 220 may include a plurality of wire portions 221 that are woven together to form a mesh structure. In an embodiment, the mesh structure formed by plurality of wire portions 221 can be configured to be suitable for a first configuration of being within the delivery catheter 510 (e.g., a compression state) and expand to a disc shape, that is, switch to the second configuration (e.g., a deployed state) when it exits from the delivery catheter 510. In some instances, the anchoring member 220 may be a single-layer mesh structure. For example, a thickness of the anchoring member 220 can be between 0.1 millimeters (mm) and 0.5 millimeters (mm). In this way, when the anchoring device 200 is configured to secure the tissue structure at the junction between the esophagus 102 and the stomach, it is beneficial to reduce the space occupied by the anchoring device 220 within the esophagus 102, so as to reduce an impact of the anchoring device 220 on eating.

In some embodiments, the anchoring members 220 of the anchoring device 200 are all formed by braiding the wire portion 221. In some embodiments, two anchoring members 220 may be an overall structure (e.g., the structure is generated using a single piece of wire). The two anchoring members 220 of the anchoring device 200 may further include a plurality of wires that are fixed to each other by welding, gluing, etc.

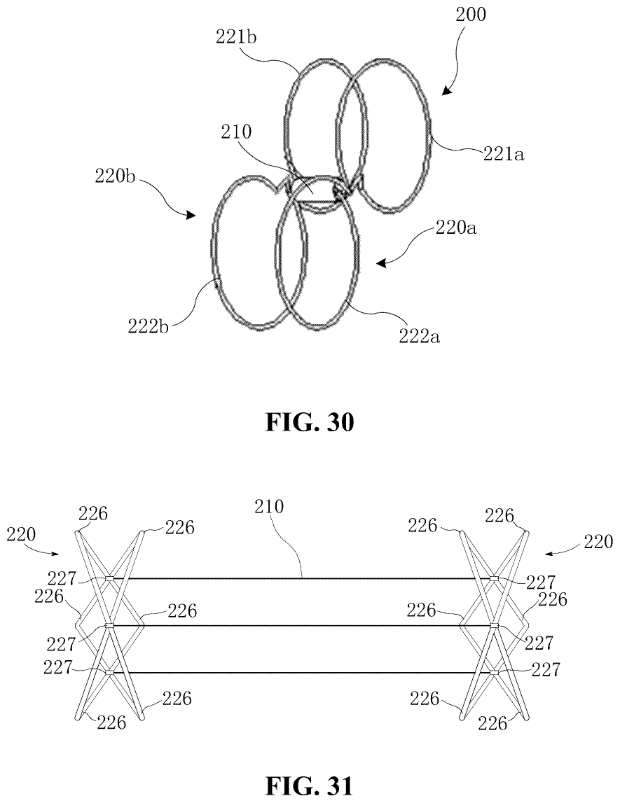

As shown in FIG. 30, in some embodiments, the shape of anchoring member 220 is similar to a “FIG. 8”. In an implementation, the anchoring member 220 includes two opposing circular portions configured to contact with one side of the target tissue.

FIG. 30 shows that the anchoring device 200 includes two anchoring members 220 and a coupling member 210 in some embodiments. One of the two anchoring members 220 is a first anchoring member 220a that is positioned at a distal end of the anchoring device 200, and the other is a second anchoring member 220b that is positioned at a proximal end of the anchoring device 200. In an embodiment, the first anchoring member 220a can be used as a gastric anchoring member for abutting the gastric fundic wall 105. The second anchoring member 220b can be used as an esophageal anchoring member for abutting the esophageal wall 104. Exemplarily, the anchoring device 200 can be delivered into the body through the delivery catheter 510 or any other suitable delivery mechanisms. Exemplarily, as shown in FIG. 30, the first anchoring member 220a includes a first sub-ring 221a and a second sub-ring 222a. In an implementation, the second anchoring member 220b includes a third sub-ring 221b and a fourth sub-ring 222b. The first anchoring member 220a and the second anchoring member 220b may be formed of wires similar to those described above. The first anchoring member 220a and the second anchoring member 220b may be an overall structure (e.g., the structure is generated using a single piece of wire). The first anchoring member 220a and the second anchoring member 220b can also include multiple wires that are fix to each other via welding, soldering, etc.

The coupling member 210 may be similar to any other coupling member described. Thus, when the first anchoring member 220a and the second anchoring member 220b are deployed, the coupling member 210 can provide a compression force so that the first anchoring member 220a and the second anchoring member 220b keep the esophageal wall 104, the left limb of right crus of diaphragm 106, and the gastric fundic wall 105 to contact with each other.

In addition to the ring pattern shown in FIG. 30, the first anchoring member 220a and the second anchoring member 220b can include any geometric configuration of wires suitable for its intended purpose. For example, the first anchoring member 220a and the second anchoring member 220b may include a shape similar to prism, oval, spiral, etc.

In some embodiments, the coupling member 210 includes a hook 211 and a traction string 212. Referring to FIGS. 13 and 14, in some embodiments, one end of the hook 211 is coupled with one of the anchoring members 220, and the other end is detachably coupled with the traction string 212. In some embodiments, the traction string 212 extends along the delivery catheter 510 towards the proximal end of the delivery catheter 510 to pull the hook 211, and then a proximal end of the hook 211 is hooked to the mesh of the anchoring member 220, thus locking the proximal-end anchoring member and the distal-end anchoring member.

In some embodiments, the hook 211 is a wire connecting the anchoring member 220 far away from the proximal end of the delivery catheter 510. In an implementation, the hook 211 can be integrated with the wire portion 221 in the anchoring member 220 far away from the proximal end of the delivery catheter 510.

In some embodiments, a central portion of the anchoring member 220 close to the proximal end of the delivery catheter 510 has a through-hole, and the hook 211 is configured to penetrate through the through-hole in the center of the anchoring member 220 close to the proximal end of the delivery catheter 510 and is coupled with the traction string 212.

Exemplarily, when two anchoring members 220 of the anchoring device 200 switch to the second configuration on both sides of the target tissue, the hook 211 can be driven by pulling the traction string 212 to render the two anchoring members 220 close to each other, so that the two anchoring members 220 in the anchoring device 200 can respectively abut against both sides of the target tissue, and then the shape of the target tissue can be reshaped through the anchoring device 200, and then the target tissue can return to normal morphology. In an implementation, the traction string 212 can be separated from the hook 211.

In some embodiments, the shape of the two anchoring members 220 in the anchoring device 200 can be arranged to be the same or different. In an implementation, the anchoring member 220 close to the proximal end of the delivery catheter 510 can be arranged as a mesh structure formed by weaving a plurality of wire portions 221. The anchoring member 220 far away from the proximal end of delivery catheter 510 can be arranged as a ring structure, such as a circular ring structure, a rectangular structure, etc.

In some embodiments, as shown in FIG. 12, the anchoring member 220 further includes a protective layer 222, which covers the wire portion 221, and the protective layer 222 blocks mesh openings formed by the wire portion 221. This embodiment is beneficial to prevent the anchoring member 220 from being embedded by the tissue, so as to facilitate to disassemble the anchoring device 200 later. Specifically, as the target tissue is easy to grow around the anchoring member 220 over time, this embodiment is beneficial to prevent the newly grown tissue structure from being embedded into the mesh.

In some embodiments, the anchoring member 220 far away from the proximal end of the delivery catheter 510 is configured as a multi-layer wire mesh structure that overlaps in a first direction. Exemplarily, when the anchoring member 220 far away from the proximal end of the delivery catheter 510 is in the second configuration, a thickness formed by the multi-layer wire mesh structure upon being deployed is greater than the thickness of the anchoring member 220 close to the proximal end of the delivery catheter 510. In some embodiments, the multi-layer wire mesh may also include a polymer coating above and/or below the mesh and mesh openings of the mesh structure are blocked by the polymer coating, when the anchoring member 220 is constructed as the multi-layer wire mesh structure.

Referring to FIGS. 7 to 12, in some embodiments, the wire portion 221 can be of a material including, but not limited to, metals, plastics, and composite materials. In some embodiments, the anchoring member 220 is constructed as a single-layer wire mesh. Of course, the anchoring member 220 can also be constructed as a multi-layer wire mesh.

In some embodiments, the wire portion 221 is of a material selected from elastic material. Specifically, the elastic deformation and folding of the wire portion 221 can be used to reduce the dimension of the anchoring member 220 in the second direction, so that the anchoring member 220 can be movably arranged within the delivery catheter 510. After the anchoring member 220 is removed from the distal end of the delivery catheter 510, the anchoring member 220 can use the elastic force of the wire portion 221 to restore the deformation to the second configuration.

In other embodiments, the wire portion 221 is made of a shape memory metal (e.g., nickel-titanium alloy), so that the anchoring member 220 can switch to the second configuration after being removed from the delivery catheter 510.

In some embodiments, the protective layer 222 is of the materials including, but not limited to, metals, plastics, and composites. Exemplarily, the protective layer 222 may be made of polymer or silica gel. In an implementation, the anchoring member 220 may be a nickel-titanium memory alloy, which may be advantageous due to its better biocompatibility and flexibility.

In some embodiments, as shown in FIGS. 7 to 9, at least one of the two anchoring members 220 is movably arranged on the coupling member 210. Exemplarily, after the two anchoring members 220 are released on both sides of the target tissue, the distance between the two anchoring members 220 may be adjusted by moving one of the anchoring members 220, so that the two anchoring members 220 may clamp and secure the tissue structure between the two anchoring members 220 to fit different tissue thickness due to individual differences.

In some embodiments, the anchoring member 220 of the two anchoring members away from the proximal end of the delivery catheter 510 is fixedly coupled with the coupling member 210, the anchoring member 220 close to the proximal end of the delivery catheter 510 is in slide-fit with the coupling member 210, so that the anchoring member 220 close to the proximal end of the delivery catheter 510 can slide along the coupling member 210. In a process of securing the target tissue, the distance between the two anchoring members 220 can be adjusted by moving the anchoring member 220 close to the proximal end of the delivery catheter 510 along an extension direction of the coupling member 210, so that the two anchoring members 220 can respectively abut against both sides of the target tissue.

In other embodiments, the coupling member 210 is a telescopic member, and the coupling member 210 extends or retracts in the first direction. In this embodiment, the distance between the two anchoring members 220 can be adjusted through extending and retracting the coupling member 210, so that the two anchoring members 220 can respectively abut against both sides of the target tissue. In an implementation, the coupling member 210 is an elastic structure, so that the coupling member 210 can be stretched or compressed as required.

In some embodiments, when the anchoring member 220 is in the second configuration, the anchoring member 220 is configured to extend and retract in the first direction and the second dimension decreases during a process of the anchoring member 220 extending in the first direction, and the second dimension increases during a process of the anchoring member 220 being compressed in the first direction. In this way, after the anchoring device 200 is anchored to the target tissue, the distance between the two anchoring members 220 can be adaptively adjusted through the extending and retracting of the anchoring members 220, so as to fit different thickness of the target tissue. In addition, during a process of the anchoring member 220 extending and retracting in the first direction, the dimension of the anchoring member 220 in the second direction can be adaptively changed to improve a stability of cooperation between the anchoring device 200 and the target tissue.

Exemplarily, the two anchoring members 220 of the anchoring device 200 are squeezed in the first direction when two parts of the anchored target tissue are separated, so that the two anchoring members 220 are compressed in the first direction. Thus, the anchoring device 200 provided by the above embodiment can adaptively increase the dimension of the two anchoring members 220 in the second direction when two parts of the anchored target tissue are separated, which is beneficial to prevent the anchoring member 220 from sliding down along a through-hole of the target tissue through which the coupling member 210 penetrates.

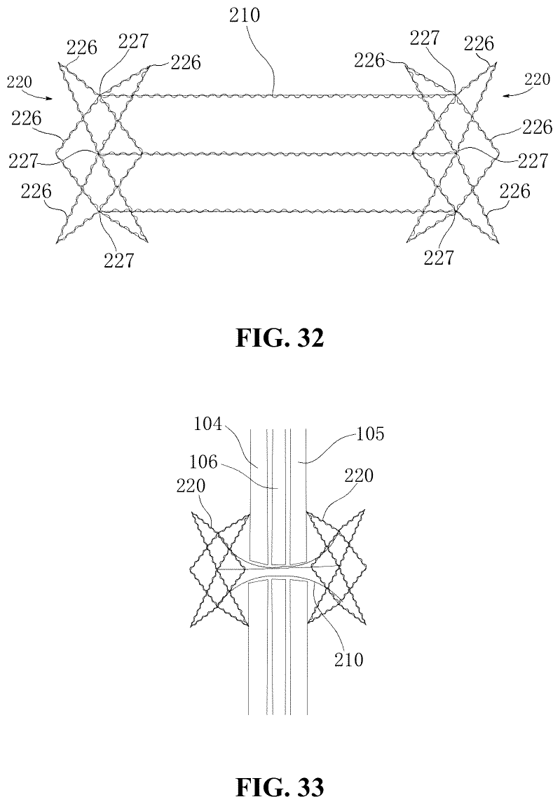

In some embodiments, as shown in FIGS. 31 and 32, the anchoring member 220 includes a plurality of anchoring feet 226, which are connected to form a ring structure. Exemplarily, the anchoring feet 226 are connected end-to-end to form a close ring shape. According to some embodiments, the plurality of anchoring feet 226 can be connected to form polygon, such as quadrilateral, pentagon, hexagon, etc. Of course, in some embodiments, the plurality of anchoring feet 226 may also be connected to form a ring, such as circular, elliptical, and semi-circular shape. Referring to FIG. 31, in some embodiments, among the multiple anchoring feet 226, each two anchoring feet 226 forms a group, the two anchoring feet 226 in each group intersect and are coupled at an intersection to form an intersection portion 227.

In some embodiments, each anchoring member 220 includes at least three groups of anchoring feet 226. Three groups of anchoring feet 226 are connected end to end to form a ring-shaped anchoring member 220. In an embodiment, the intersection portion 227 formed at the intersection of each group of anchoring feet 226 is connected to an end portion of the coupling member 210.

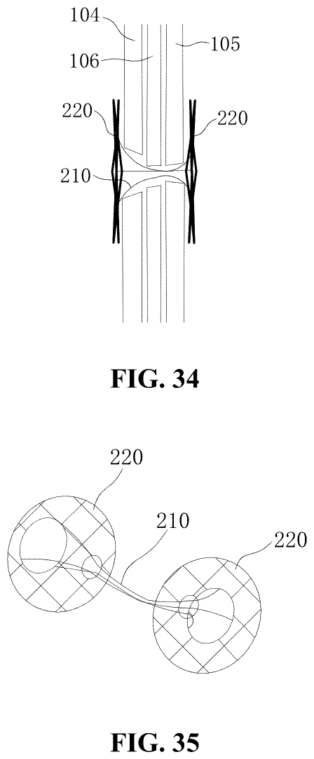

Referring to FIGS. 33 and 34, in some embodiments, when the dimension of the target tissue between the two anchoring members 220 increases in the first direction, the two anchoring members 220 deform under an extrusion of the target tissue, thereby reducing an included angle between the two anchoring feet 226 in at least one group of anchoring feet 226, and realizing the contracting of the anchoring member 220 in the first direction, and increasing the dimension of the anchoring member 220 in the second direction.

In other embodiments, referring to FIGS. 35 and 36, the anchoring member 220 may be a spherical shell structure.

According to some embodiments, the two anchoring members 220 in the anchoring device 200 are each provided as a spherical shell structure, opposite sides of the two anchoring members 220 in the first direction are each provided with an opening. The coupling member 210 penetrates through the opening on the anchoring member 220 and is connected with one side of the anchoring member 220 that is opposite to the opening. Exemplarily, a side of the distal-end anchoring member 220 close to the proximal-end anchoring member 220 is provided with an opening, and a side of the proximal-end anchoring member 220 close to the distal-end anchoring member 220 is provided with an opening. The coupling member 210 penetrates through the opening of the distal-end anchoring member 220 and is connected to a side of the distal-end anchoring member 220 away from the proximal-end anchoring member 220. The coupling member 210 penetrates through the opening of the proximal-end anchoring member 220 and is connected to a side of the proximal-end anchoring member 220 away from the distal-end anchoring member 220. In this way, in a case where the thickness of the target tissue between the two anchoring members 220 increases, the anchoring member 220 is compressed in the first direction, and the dimension of the anchoring member 220 in the second direction increases.

According to some optional embodiments, the distal-end anchoring member 220 is a spherical shell structure. The proximal-end anchoring member 220 may be a circular or a disc-shaped structure. In this way, the anchoring device 200 can not only adapt to the size of the target tissue between two anchoring components 220 through a deformation of the distal-end anchoring member 220, but also ensure that the proximal-end circular or disc-shaped structure does not obstruct the food smoothly passing through the esophagus.

In some embodiments, in the above embodiments, both ends of the coupling member 210 can be fixedly coupled with two anchoring members 220, respectively.

In some embodiments, the present application provides an anchoring apparatus for treating a loss of function of the anti-reflux valve at the junction between esophagus 102 and gastric fundus 103 by recovering a relationship between the esophageal wall 104, diaphragmatic muscle, and gastric wall. While disclosed with specific reference to the treatment referenced above, it should be readily understood that the anchoring apparatus described in the present application can also be used in other procedures.

In some embodiments, one of the application scenarios of the anchoring apparatus provided in the present application is for a gastrointestinal procedure. Exemplarily, one of the two anchoring members 220 of the anchoring device 200 is the gastric anchoring member and the other is the esophageal anchoring member. Exemplarily, the anchoring apparatus includes the delivery catheter 510, the gastric anchoring member, the esophageal anchoring member, and the coupling member 210 extending between the gastric anchoring member and the esophageal anchoring member. The gastric anchoring member and the esophageal anchoring member are configured to move to a distal end of the delivery catheter 510 relative to the delivery catheter 510 in one configuration, and switch to another configuration after leaving the delivery catheter 510. The gastric anchoring member and the esophageal anchoring member render the esophageal wall, diaphragm muscle, and gastric wall to be in contact with each other to restore the function of the anti-reflux valve.

In some embodiments, the anchoring device 200 is configured to anchor the target tissue. As an exemplarily structure, the target tissue can be a tissue structure that needs to be restored to a normal morphology. Exemplarily, the target tissue includes an esophageal wall 104, a left limb of right crus of diaphragm 106, and a gastric fundic wall 105. Among them, the left limb of right crus of diaphragm 106 is positioned between the esophageal wall 104 and the gastric fundic wall 105, so that the esophageal wall 104 and the gastric fundic wall 105 can form a His angle.

In some embodiments, before the anchoring device 200 is anchored to the target tissue, an included angle between a medial surface of a target area of the esophageal wall 104 and a medial surface of the target area of the gastric fundic wall 105 is a first included angle. Exemplarily, the first included angle can be His angle after the cardiac orifice loses its anti-reflux function. Exemplarily, when the His angle increases, the anti-reflux mechanism will become ineffective and the contents of the stomach will easily form reflux, thereby causing GERD.

Referring to FIG. 25, after the anchoring device 200 is secured to the target tissue, the coupling member 210 penetrates through the esophageal wall 104, the left crus of diaphragm, and the gastric fundic wall 105. One of the two anchoring members 220 is configured to abut against the medial surface of the target area of the gastric fundic wall 105, and the other is configured to abut against the medial surface of the target area of the esophageal wall 104. An included angle between the medial surface of the target area of the gastric fundic wall 105 and the medial surface of the target area of the esophageal wall 104 is a second included angle, the second included angle is smaller than the first included angle.

The anchoring device 200 provided by the above embodiment is beneficial to remold the target tissue structure, reduce the His angle formed by the esophageal wall 104 and the gastric fundic wall 105, and then can be used to secure the gastric wall, diaphragm muscle and esophageal wall, and restore the anti-reflux function at the junction between the esophagus and stomach. Exemplarily, the second included angle is an acute angle.

Referring to FIG. 26, in some embodiments, after the anchoring device 200 is secured to the target tissue, the target tissue forms a piercing hole, the coupling member 210 penetrates through the esophageal wall 104, the left crus of diaphragm, and the gastric fundic wall 105 along the piercing hole, and the anchoring device 200 seals the piercing hole. Exemplarily, after the anchoring device 200 is secured to the target tissue, the anchoring device 200 compresses the piercing hole to be a closed state. In an implementation, after the anchoring device 200 is secured to the target tissue, at least one anchoring member 220 in the anchoring device 200 closes a port of the piercing hole. In some embodiments, after the anchoring device 200 is secured to the target tissue, a distal-end anchoring member in the anchoring device 200 closes the port of the piercing hole.

Exemplarily, a dimension of at least one of the two anchoring members 220 of the anchoring device 200 in a direction perpendicular to an extension direction of the coupling member 210 is greater than a dimension of the piercing hole in a direction perpendicular to the extension direction of the coupling member 210. In this way, the piercing hole can be sealed by the anchoring member 220.

In some embodiments, as shown in FIGS. 2 to 6, one of two anchoring members 220 in the anchoring device 200 is a gastric anchoring member, and the other is an esophageal anchoring member. In an implementation, the gastric anchoring member is an anchoring member 220 far away from the proximal end of the delivery catheter 510. The esophageal anchoring member is an anchoring member 220 close to the proximal end of the delivery catheter 510. The gastric anchoring member, the esophageal anchoring member, and the coupling member 210 are all configured to fit within the catheter (not shown) for accessing the patient's esophagus. Therefore, the gastric anchoring member and the esophageal anchoring member can have a variety of shape configurations. For example, the gastric anchoring member can have a first configuration. The gastric anchoring member is configured to fit in the delivery catheter 510 when the gastric anchoring member is in the first configuration, that is, the gastric anchoring member can be provided in the delivery catheter 510. The gastric anchoring member can have a second configuration. The gastric anchoring member will not be suitable to be positioned in the delivery catheter 510 when the gastric anchoring member is in the second configuration. Exemplarily, when the gastric anchoring member is in the second configuration, a dimension of the gastric anchoring member in the second direction is larger than an inner diameter of the delivery catheter 510. In an implementation, when the gastric anchoring member is in the second configuration, the dimension of the gastric anchoring member in the second direction is larger than the outer diameter of the delivery catheter 510.

Exemplarily, the esophageal anchoring member may have a first configuration. The esophageal anchoring member is configured to fit in the delivery catheter 510 when the esophageal anchoring member is in the first configuration. The esophageal anchoring member may also have a second configuration. The esophageal anchoring member will not fit in the catheter when the esophageal anchoring member is in the second configuration. Exemplarily, when the esophageal anchoring member is in the second configuration, the dimension of the esophageal anchoring member in the second direction is larger than an inner diameter of the delivery catheter 510. In an implementation, when the esophageal anchoring member is in the second configuration, the dimension of the esophageal anchoring member in the second direction is larger than the outer diameter of the delivery catheter 510.

In operation, the delivery catheter 510 extends through the esophageal wall 104, the left limb of right crus of diaphragm 106, and the gastric fundic wall 105. The anchoring device 200 is inserted via the delivery catheter 510 until the anchoring member 220 at the distal end extends to the distal end of the delivery catheter 510 and to the gastric fundus 103. The delivery catheter 510 is further retracted back towards the proximal end to release the anchoring member 220 at the distal end in the delivery catheter 510, that is, the gastric anchoring member, so that the distal-end anchoring member 220 can switch to the second configuration. After the distal-end anchoring member 220 abuts against the gastric fundic wall 105, the delivery catheter 510 is further retracted back towards the proximal end to release the proximal-end anchoring member 220, that is, the esophageal anchoring member, so that the proximal-end anchoring member 220 can switch to the second configuration.

In some embodiments, the proximal-end anchoring member 220 and the distal-end anchoring member 220 can be adjusted through the contraction of the coupling member 210, so that the distal-end anchoring member 220 can abut against the gastric fundic wall 105, and the proximal-end anchoring member 220 can abut against the esophageal wall 104, so that an included angle between the esophagus 102 and the gastric fundus 103 can be restored to be normal, and thus the anti-reflux effect can be achieved at the junction between the esophagus 102 and the gastric fundus 103.

In some embodiments, one of the two anchoring members 220 is fixedly coupled with a first end of the coupling member 210, and the other has a first through-hole 223. A second end of the coupling member 210 passes through the first through-hole 223 and is in slide-fit with the second anchoring member 220b.

As shown in FIGS. 7 to 9, one of the two anchoring members 220 is the first anchoring member 220a and the other is the second anchoring member 220b. The first anchoring member 220a is fixedly coupled with a first end of the coupling member 210. The second anchoring member 220b has a first through-hole 223, where the second end of the coupling member 210 passes through the first through-hole 223 and is in slide-fit with the second anchoring member 220b.

In the above embodiment, the second anchoring member 220b can slide along the coupling member 210 such that the second anchoring member 220b can move towards or away from the first anchoring member 220a. In a process of securing the target tissue, the distance between the first anchoring member 220a and the second anchoring member 220b can be adjusted by operating the second anchoring member 220b to slide along the coupling member 210, so that the first anchoring member 220a and the second anchoring member 220b can abut against two different sides of the target tissue respectively.

In some embodiments, the first anchoring member 220a is an anchoring member 220 far away from the proximal end of the delivery catheter 510. The second anchoring member 220b is an anchoring member 220 close to the proximal end of the delivery catheter 510. In this way, it is beneficial to reduce a difficulty of adjusting a movement of the second anchoring member 220b relative to the coupling member 210.

In an implementation, when the anchoring device 200 is configured to secure the tissue structure at the junction between the esophagus 102 and the gastric fundus 103, the first anchoring member 220a can be used as the gastric anchoring member to abut the gastric fundic wall 105 via the first anchoring member 220a. The second anchoring member 220b can be used as an esophageal anchoring member to abut the esophageal wall 104 through adjusting the relative position between the second anchoring member 220b and the coupling member 210, and the two anchoring members render the junction of the gastric fundus 103 restores to an angle with anti-reflux function during a process of abutting.

In some embodiments, as shown in FIGS. 7, 21 and 23, the anchoring apparatus provided by the present application further includes a locking mechanism 300. Exemplarily, the locking mechanism 300 can be configured to limit the second anchoring member 220b to slide relative to the coupling member 210. In some embodiments, the locking mechanism 300 cooperates with the coupling member 210, and is configured to switch between an unlocking state and a locking state. When the locking mechanism 300 is in the unlocking state, the locking mechanism 300 is in slide-fit with the coupling member 210, and when the locking mechanism 300 is in the locking state, the locking mechanism 300 is in limiting-fit with the coupling member 210 in an extension direction of the coupling member 210.

Exemplarily, in a process of securing the target tissue, the locking mechanism 300 is first sleeved on the coupling member 210, and then is delivered to the target tissue along the forceps channel of the endoscope 100. By pulling an external portion of the coupling member 210, the first anchoring member 220a abuts against an internal face of the stomach, and the locking mechanism 300 is used to push the second anchoring member 220b toward the distal end, so that the second anchoring member 220b is tightly close to the internal face of the esophagus, thereby adjusting the distance between the first anchoring member 220a and the second anchoring member 220b. Thus, in this embodiment, the locking mechanism 300 can cause the first anchoring member 220a and the second anchoring member 220b to fully compress the target tissue between the two anchoring members 220, and a recovery situation of the target tissue morphology can be observed through the endoscope 100 (e.g., observing a recovery situation of the His angle). Upon confirming that the therapeutic effect is achieved, that is, when the target tissue morphology returns to normal, the second anchoring member 220b and the coupling member 210 are locked by the locking mechanism 300 to prevent the second anchoring member 220b from sliding relative to the coupling member 210, and an extra portion of the coupling member 210 is further cut through the locking mechanism 300 to complete an placement of the anchoring device 200.

In some embodiments, a lateral wall of the delivery catheter 510 is provided with a wire-through hole, and the coupling member 210 runs out of the delivery catheter 510 through the wire-through hole, so as to separate the coupling member 210 from the delivery device 500. Exemplarily, the wire-through hole is arranged close to the proximal end of the delivery catheter 510. In some embodiments, a length of the delivery device 500 passing through the target tissue can be determined by a relative position between the wire-through hole and the forceps channel in the endoscope 100, so as to ensure that the distal-end anchoring member of the anchoring device 200 completely passes through the target tissue during the placement of the anchoring device 200 in the target tissue.

In some embodiments, the locking mechanism 300 is arranged at one side of the second anchoring member 220b away from the first anchoring member 220a. In an implementation, a dimension of the locking mechanism 300 in a radial direction of the coupling member 210 is larger than a hole diameter of the hole in the second anchoring member 220b for the coupling member 210 to pass through, so that the locking mechanism 300 can limit the second anchoring member 220b to slide relative to the coupling member 210 by abutting against one side of the second anchoring member 220b that is far away from the first anchoring member 220a.

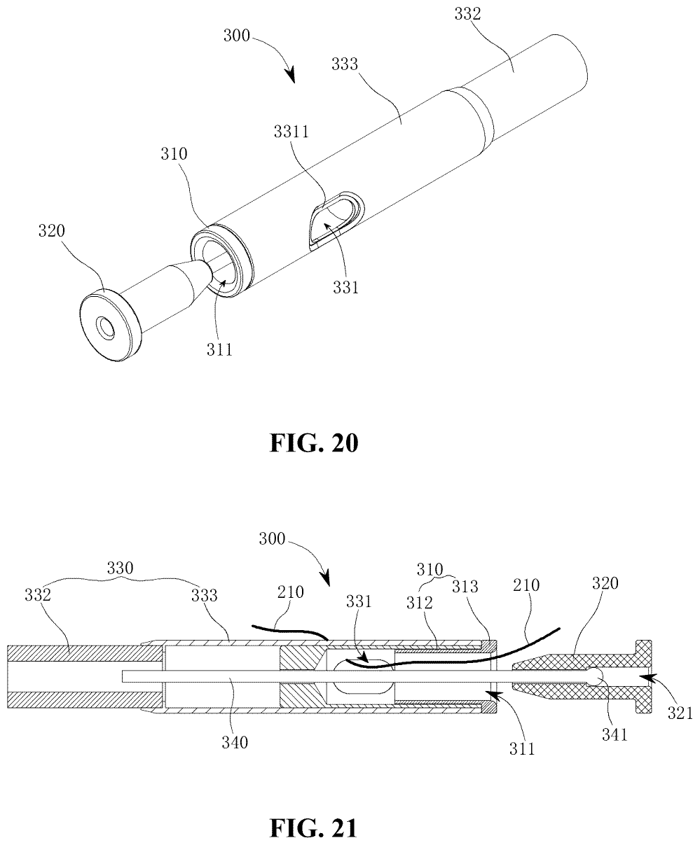

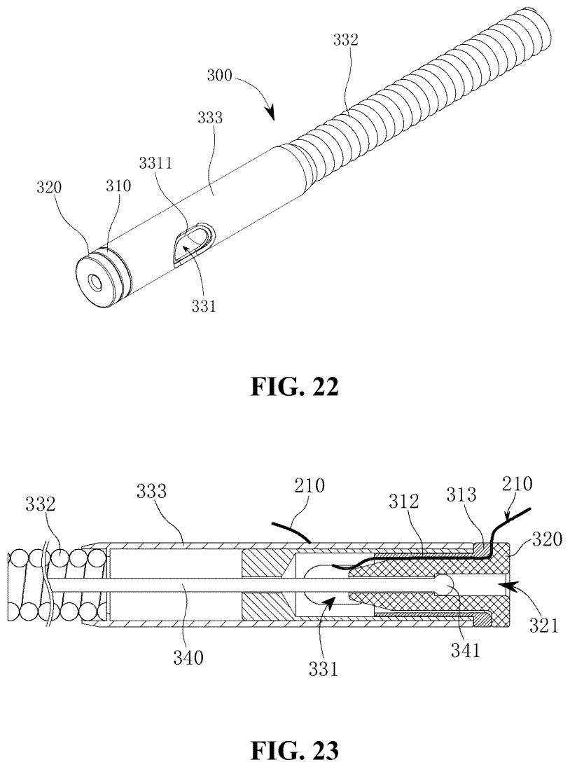

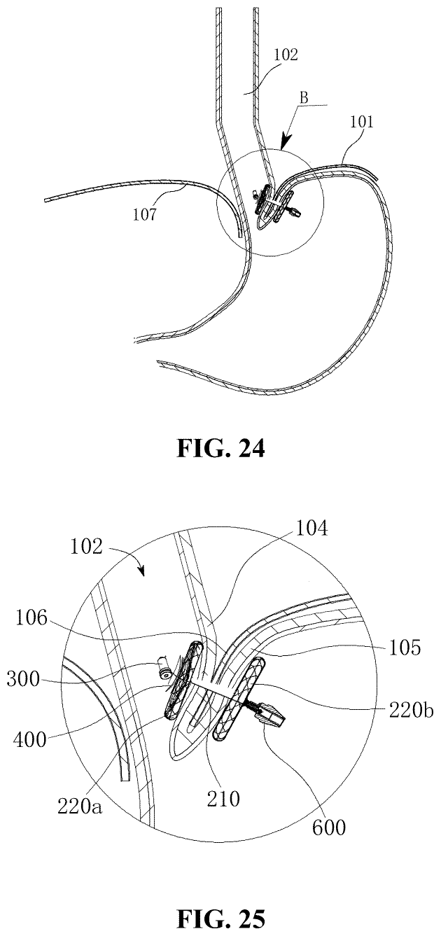

In some embodiments, as shown in FIGS. 20 to 23, the locking mechanism 300 includes a first locking member 310 and a second locking member 320, the first locking member 310 has a first mounting hole 311, and the coupling member 210 passes through the first mounting hole 311.

When the locking mechanism 300 is in the unlocking state, a first gap is formed between the second locking member 320 and the first locking member 310, and the coupling member 210 passes through the first gap and is in slide-fit with at least one of the first locking member 310 and the second locking member 320. Exemplarily, a width of the first gap is larger than a diameter of the coupling member 210, so that the coupling member 210 can slide along the first gap.

When the locking mechanism 300 is in the locking state, at least part of the second locking member 320 is positioned in the first mounting hole 311, and the second locking member 320 and the first locking member 310 respectively abut against different locations of the coupling member 210 and are fixed on the coupling member 210. Exemplarily, when the second locking member 320 is embedded in the first mounting hole 311, an outer lateral wall of the second locking member 320 and an inner side of the first mounting hole 311 can respectively abut against two radially opposite sides of the coupling member 210, so that the first locking member 310 and the second locking member 320 can be clamped and fixed to the coupling member 210. That is, when the locking mechanism 300 is in the locking state, the locking mechanism 300 can abut against the second anchoring member 220b to limit the second anchoring member 220b to slide relative to the coupling member 210.

In some embodiments, the first locking member 310 and the second locking member 320 can select a material with a higher friction coefficient. Of course, in some embodiments, one of the first locking member 310 and the second locking member 320 may be provided with a bulge portion, and the other may be provided with a concave portion. When the locking mechanism 300 is in the locking state, the bulge portion and the concave portion fit with each other, which is beneficial to improve a reliability of the locking mechanism 300 and the coupling member 210 after being locked.

Referring to FIGS. 20 to 22, in some embodiments, the locking mechanism 300 further includes a first catheter 330 and a first mandrel 340. The first mandrel 340 is movably arranged within the first catheter 330. In the embodiment, the first mandrel 340 passes through the first catheter 330. Exemplarily, the first mandrel 340 is in clearance-fit with the first catheter 330, that is, an outer diameter of the first mandrel 340 is smaller than an inner diameter of the first catheter 330 such that the first mandrel 340 can move along the first catheter 330.

In some embodiments, the first mandrel 340 penetrates through the first catheter 330 and is detachably coupled with the second locking member 320. In an implementation, one end of the first catheter 330 close to an operator is the proximal end of the first catheter 330. The other end of the first catheter 330 far away from the operator is the distal end of the first catheter 330. In an embodiment, one end of the first mandrel 340 coupling with the second locking member 320 runs out of the distal end of the first catheter 330 and is coupled with the second locking member 320. In an implementation, one end of the first mandrel 340 far away from the second locking member 320, namely the proximal end of the first mandrel 340, penetrates through the proximal end of the first catheter 330, so that the first mandrel 340 can move relative to the first catheter 330 by operating the proximal end of the first mandrel 340 via an operator, and then the second locking member 320 is driven to move relative to the first catheter 330.

In some embodiments, the first locking member 310 is sleeved on the first mandrel 340 and is in clearance-fit with the first mandrel 340 to cause the first mandrel 340 to slide relative to the first locking member 310. The first locking member 310 is positioned between the second locking member 320 and the first catheter 330. At least part of the first locking member 310 abuts against an end surface of one end of the first catheter 330 close to the second locking member 320, that is, at least part of the first locking member 310 abuts against the distal end of the first catheter 330.

Referring to FIGS. 20 and 21, upon the first anchoring member 220a and the second anchoring member 220b respectively abutting against two sides of the target tissue, the delivery catheter 510 can be taken out. Then, the proximal end of the coupling member 210 penetrates through the first locking member 310 along a first gap formed between the first locking member 310 and the second locking member 320.

In an implementation, by operating synchronously the first catheter 330 and the first mandrel 340, the first catheter 330 and the first mandrel 340 can make the first locking member 310 and the second locking member 320 move to a location close to the second anchoring member 220b along an extension direction of the coupling member 210.

In an implementation, the first catheter 330 can support the fist locking member 310 by the first catheter 330 supporting the first locking member 310 and pushing the proximal end of the coupling member 210, and the second anchoring member 220b and the first anchoring member 220 are close to each other by pushing the second anchoring member 220b to move relative to the coupling member 210 via the first locking member 310.

Upon the second anchoring member 220b and the first anchoring member 220a respectively abutting against both sides of the target tissue and the morphology of the secured tissue structure returning to normal, the proximal end of the first mandrel 340 is pulled to make the first mandrel 340 move towards a direction close to the proximal end relative to the first catheter 330, and then the second locking member 320 is driven to move relative to the first locking member 310.

In an implementation, the first mandrel 340 is pulled such that at least part of the second locking member 320 is positioned in the first mounting hole 311 of the first locking member 310 and abuts against the first locking member 310 at different locations of the coupling member 210, thereby realizing that the locking mechanism 300 is switched to the locking state from the unlocking state.

In some embodiments, the first mandrel 340 is clamped with the second locking member 320, and the first mandrel 340 can be separated from the second locking member 320 by directly dragging the first mandrel 340.

Referring to FIGS. 21 and 23, in some embodiments, one end of the first mandrel 340 coupling with the second locking member 320 is provided with a buckle portion 341, one end of the second locking member 320 close to the first mandrel 340 is provided with a buckle groove 321, and at least part of the buckle portion 341 is positioned in the buckle groove 321 and is fastened with the second locking member 320. In an implementation, at least part of the second locking member 320 is made of an elastic material. In a process of dragging the first mandrel 340, as at least part of the second locking member 320 abuts against the first locking member 310, the second locking member 320 can deform by increasing the force of dragging the first mandrel 340, and then the second locking member 320 can slide out of the buckle groove 321 to realize that the first mandrel 340 is detached from the second locking member 320.

Of course, in other embodiments, the first mandrel 340 is coupled with the second locking member 320 via a threaded connection. Specifically, the first mandrel 340 can be separated from the second locking member 320 by rotating the first mandrel 340. In an implementation, one of the first locking member 310 and the second locking member 320 is provided with a limit groove, and the other is provided with a limit protrusion. When the locking mechanism 300 is in the locking state, at least part of the limit protrusion is positioned within the limit groove to limit the second locking member 320 to rotate relative to the first locking member 310, which is beneficial to reduce the difficulty of disassembling the screw-thread fit between the first mandrel 340 and the second locking member 320.

Referring to FIGS. 20 to 23, in some embodiments, the first locking portion 310 has a positioning portion 312 and a limit portion 313, at least part of the positioning portion 312 is positioned within the first catheter 330, the limit portion 313 protrudes from an outer peripheral wall of the positioning portion 312, and at least part of the limit portion 313 abuts an end surface of one end of the first catheter 330 close to the second locking member 320.

In the above embodiment, at least part of the positioning portion 312 is positioned within the first catheter 330, which can define the configuration of the first locking member 310 relative to the second locking member 320 through the first catheter 330, thus, it is benefit to reduce the difficulty of the second locking member 320 being embedded into the first mounting hole 311 of the first locking member 310, and further reduce the difficulty of the locking mechanism 300 switching from the unlocking state to the locking state.

Upon the locking mechanism 300 switching to the locking state, the first mandrel 340 is first detached from the second locking member 320, and then the first catheter 330 may be separated from the first locking member 310 by pulling the first catheter 330.

In some embodiments, as shown in FIGS. 20 to 23, a lateral wall of one end of the first catheter 330 close to the second locking member 320 has a cutting port 331, the cutting port 331 penetrates through the lateral wall of the first catheter 330 and is communicated with the first catheter 330, and a cutting edge 3311 is arranged along an edge of the cutting port 331. The coupling member 210 penetrates into the first catheter 330 from one end of the first catheter 330 close to the second locking member 320, runs out of the first catheter 330 from the cutting port 331, and extends along the first catheter 330 towards one end far away from the second locking member 320. In this way, the coupling member 210 can be cut through the cutting edge 3311 at the cutting port 331.

In some embodiments, the cutting edge 3311 can be provided as a sharp blade, so that the cutting edge 3311 can cut the coupling member 210 in the process of pulling the first catheter 330, and then cut the coupling member 210 from a location adjacent to the locking mechanism 300.

In some embodiments, as shown in FIGS. 20 to 23, the first catheter 330 includes a catheter body 332 and a cutting head 333. In an implementation, the cutting head 333 is arranged at a distal end of the catheter body 332, and is communicated with the catheter body 332, so that the first mandrel 340 can penetrate through the cutting head 333 and be coupled with the second locking member 320. In an embodiment, the cutting port 331 is arranged at a lateral wall of the cutting head 333. In an implementation, the cutting head 333 can be made of a hard material, such as metal, ceramics, etc.

In some embodiments, the material of the catheter body 332 may be, but not limited to, metal or composite materials. In some embodiments, the catheter body 332 can be provided as a metal spring, so that the catheter body 332 can provide sufficient support force in its extension direction, and have better flexibility in its radial direction, so as to facilitate to penetrate in an internal cavity of the body.

Referring to FIGS. 7 to 9, 18 and 19, in some embodiments, the anchoring device 200 further includes a skirt-side pad 400. In some embodiments, the skirt-side pad 400 is positioned on at least one side of the anchoring member 220 in the first direction. In an embodiment, the skirt-side pad 400 is movably arranged on the coupling member 210. Exemplarily, the skirt-side pad 400 includes at least two through-holes 410 distributed along an extension direction of the skirt-side pad 400, the through-holes 410 penetrate through the skirt-side pad 400 along a direction of the thickness of the skirt-side pad 400, and the coupling member 210 is successively inserted into the skirt-side pad 400 along a plurality of through-holes 410 and is in slide-fit with the through-holes 410. Exemplarily, the coupling member 210 can be repeatedly inserted into the skirt-side pad 400 through the plurality of through-holes 410, so that the skirt-side pad 400 can be folded after the coupling member 210 is tightened.

Exemplarily, when the locking mechanism 300 is assembled on the anchoring device 200, the skirt-side pad 400 is positioned between the second anchoring member 220b and the locking mechanism 300, which can prevent the locking mechanism 300 from directly abutting on the proximal-end anchoring member, which is beneficial to protect the proximal-end anchoring member.

In some embodiments, the skirt-side pad 400 is positioned between two anchoring members 220. Exemplarily, when the anchoring device 200 is placed into the target tissue, the skirt-side pad 400 is positioned between the anchoring member 220 and the target tissue.

In some embodiments, there are two skirt-side pads 400 between the two anchoring members 220. Exemplarily, when the anchoring device 200 is placed into the target tissue, one of the two skirt-side pads 400 between the two anchoring members 220 is positioned between the distal-end anchoring member of the anchoring device 200 and the target tissue, and the other is positioned between the proximal-end anchoring member of the anchoring device 200 and the target tissue.

In some embodiments, the anchoring apparatus further includes a plurality of skirt-side pads 400, and the skirt-side pads 400 are each disposed between the second anchoring member 220b and the locking mechanism 300 and between the two anchoring members 220.

In an implementation, the skirt-side pad 400 can be made of a flexible material. Exemplarily, in the process of the anchoring device 200 securing the target tissue, one end of the skirt-side pad 400 far away from the second anchoring member 220b is pushed by operating the locking mechanism 300, so that the skirt-side pad 400 can slide along the coupling member 210, and the skirt-side pad 400 can be squeezed and folded to form a multi-layer flexible pad. This embodiment is beneficial to prevent the locking mechanism 300 from directly squeezing the second anchoring member 220b, and thus to protect the second anchoring member 220b. Upon the locking mechanism 300 being released, a stress concentration formed by the locking mechanism 300 on the second anchoring member 220b can be minimized, and the stress can be dispersed, thereby preventing a risk of wire breakage, damage and other risks caused by the long-term stress concentration of the second anchoring member 220b. In some embodiments, both sides of the second anchoring member 220b and the first anchoring member 220a can be attached with a flexible skirt to reduce the compressive stress on the tissue. In some embodiments, the material of skirt-side pad 400 can be a flexible polymer material. In an implementation, the skirt-side pad 400 can be made of corrosion-resistant e-PTFE (expanded polytetrafluoroethylene), or PU (poly urethane), TPU (Thermoplastic Urethane), PE (polyethylene) film, etc.

While the disclosure has been described in connection with certain embodiments, it is to be understood that the disclosure is not to be limited to the disclosed embodiments but, on the contrary, is intended to cover various modifications and equivalent arrangements included within the scope of the appended claims. This scope is to be accorded the broadest interpretation so as to encompass all such modifications and equivalent structures as is permitted under the law.

Finally, it should be noted that the above embodiments are only used to illustrate the technical solutions of the present application, not to limit it. Although the present application has been described in detail with reference to the preceding embodiments, ordinary technicians in the art should understand that they can still modify the technical solutions recorded in the preceding embodiments, or substitute some or all of the technical features equivalently. However, these modifications or substitution do not make the essence of the corresponding technical solutions depart from the scope of the technical solutions of the embodiments of the present application.

What is claimed is:

1. An anchoring device, wherein the anchoring device comprises a coupling member and two anchoring members, the two anchoring members are arranged on the coupling member at intervals along a first direction, a dimension of the coupling member in a second direction is a first dimension, and the second direction is perpendicular to the first direction;
the anchoring member is configured to switch between a first configuration and a second configuration, and when the anchoring member is in the first configuration, a dimension of the anchoring member in a direction perpendicular to the first direction is a second dimension;
when the anchoring member is in the second configuration, a dimension of the anchoring member in the second direction is a third dimension, and the third dimension is greater than the second dimension and the first dimension.

2. The anchoring device according to claim 1, wherein the anchoring member comprises a wire portion, and the wire portion is configured to switch between a compression state and a deployed state,
the anchoring member is in the first configuration when the wire portion is in the compression state,
the anchoring member is in the second configuration when the wire portion is in the deployed state.

3. The anchoring device according to claim 2, wherein the wire portion forms a foldable mesh structure;
wherein the anchoring member further comprises a protective layer, the protective layer covers the wire portion and blocks mesh openings formed by the wire portion.

4. The anchoring device according to claim 2, wherein the wire portion is made of an elastic material; or the wire portion is made of a shape memory metal.

5. The anchoring device according to claim 1, wherein at least one of the two anchoring members is movably arranged on the coupling member; or
the coupling member is a telescopic member, and the coupling member extends or retracts in the first direction.

6. The anchoring device according to claim 1, wherein when the anchoring member is in the second configuration, the anchoring member is configured to extend and retract in the first direction, and the second dimension decreases during a process of the anchoring member extending in the first direction, and the second dimension increases during a process the anchoring member being compressed in the first direction.

7. The anchoring device according to claim 1, wherein the coupling member is flexibly coupled with the anchoring member;
wherein the coupling member is a flexible coupling wire.

8. The anchoring device according to claim 1, wherein the anchoring device is configured to anchor a target tissue; the target tissue comprises an esophageal wall, a left limb of right crus of diaphragm and a gastric fundic wall, the left limb of right crus of diaphragm is positioned between the esophageal wall and the gastric fundic wall;
before the anchoring device is anchored to the target tissue, an included angle between a medial surface of a target area of the esophageal wall and a medial surface of a target area of the gastric fundic wall is a first included angle;
after the anchoring device is secured to the target tissue, the target tissue forms a piercing hole, and the coupling member penetrates through the esophageal wall, the left limb of right crus of diaphragm, and the gastric fundic wall along the piercing hole; one of the two anchoring members is configured to abut against the medial surface of the target area of the gastric fundic wall, and the other is configured to abut against the medial surface of the target area of the esophageal wall, an included angle between the medial surface of the target area of the gastric fundic wall and the medial surface of the target area of the esophageal wall is a second included angle, the second included angle is smaller than the first included angle.

9. The anchoring device according to claim 8, wherein at least one anchoring member in the anchoring device closes a port of the piercing hole after the anchoring device is secured to the target tissue.

10. An anchoring apparatus, comprising a delivery device and the anchoring device according to claim 1, wherein the delivery device comprises a delivery catheter, the anchoring device is movably arranged within the delivery catheter and is configured to thread out of one end of the delivery catheter;
when the anchoring device is positioned within the delivery catheter, the anchoring member is in the first configuration;
when the anchoring device is positioned outside the delivery catheter, the anchoring member is in the second configuration.

11. The anchoring apparatus according to claim 10, wherein an outer diameter of the delivery catheter is a fourth dimension, the third dimension is larger than the fourth dimension.

12. The anchoring apparatus according to claim 10, further comprising a piercing head, wherein the piercing head comprises a thermal-insulation member and a conductor, and the conductor is arranged on the thermal-insulation member.

13. The anchoring apparatus according to claim 12, wherein the piercing head is arranged at a distal end of the delivery catheter or the anchoring device, the piercing head is configured to penetrate through a structure of an anchored tissue.

14. The anchoring apparatus according to claim 12, wherein the piercing head has a first avoidance hole that penetrates through the piercing head, the anchoring device is configured to extend out of the first avoidance hole.

15. The anchoring apparatus according to claim 12, wherein the delivery device further comprises a second mandrel, at least part of the second mandrel is positioned within the delivery catheter, the second mandrel is configured to be movable relative to the delivery catheter.

16. The anchoring apparatus according to claim 15, wherein a first end of the second mandrel is detachably coupled with the anchoring device; or a first end of the second mandrel is fixedly or detachably coupled with the piercing head.

17. The anchoring apparatus according to claim 15, wherein the delivery device further comprises a limiting member arranged on the second mandrel, the limiting member is positioned at one side of the delivery catheter far away from the anchoring device,
the limiting member is configured to switch between a first state and a second state, when the limiting member is in the first state, the limiting member is in limiting-fit with the delivery catheter in an extension direction of the delivery catheter;
when the limiting member is in the second state, the limiting member is in slide-fit with the delivery catheter, and the limiting member slides along the extension direction of the delivery catheter.

18. The anchoring apparatus according to claim 15, wherein an assembly gap is disposed between the second mandrel and the delivery catheter; when the anchoring device is positioned within the delivery catheter, the anchoring member is positioned between the second mandrel and the delivery catheter.

19. The anchoring apparatus according to claim 10, wherein the delivery device further comprises a third mandrel, the third mandrel comprises a fourth catheter, a booster and a puncture needle, a first end of the puncture needle is a tip, a second end of the puncture needle is coupled with the fourth catheter, the puncture needle has a needle hole that penetrates from the first end of the puncture needle to the second end of the puncture needle and is communicated with the fourth catheter,
the anchoring device is movably arranged in the fourth catheter or the puncture needle, at least part of the booster is positioned in the fourth catheter or the puncture needle, the booster is configured to be movable relative to the fourth catheter and the puncture needle;
the booster abuts the anchoring device, and is configured to push the anchoring device out of the puncture needle.

20. The anchoring apparatus according to claim 10, wherein one of the two anchoring members is fixedly coupled with a first end of the coupling member, the other has a first through-hole, the second end of the coupling member passes through the first through-hole and is in slide-fit with the anchoring member;
the anchoring apparatus further comprises a locking mechanism cooperating with the coupling member, the locking mechanism is configured to switch between an unlocking state and a locking state; when the locking mechanism is in the unlocking state, the locking mechanism is in slide-fit with the coupling member; when the locking mechanism is in the locking state, the locking mechanism is in limiting-fit with the coupling member in an extension direction of the coupling member.
